(19) United States
(12) Patent Application Publication
(10) Pub. No.: US 2024/0186014 A1
(43) Pub. Date: Jun. 6, 2024

(54) SYSTEMS AND METHODS FOR PREDICTING BRAIN BIOMARKER STATUS

(71) Applicant: COGNETIVITY LTD, Holborn London, Greater London (GB)

(72) Inventors: Seyed-Mahdi KHALIGH-RAZAVI, Holborn London, Greater London (GB); Chris KALAFATIS, Holborn London, Greater London (GB); Mohammad Hadi MODARRES, Holborn London, Greater London (GB); Jonathan EL-SHARKAWY, Holborn London, Greater London (GB); Sina HABIBI, Holborn London, Greater London (GB)

(21) Appl. No.: 18/285,371

(22) PCT Filed: Mar. 30, 2022
(86) PCT No.: PCT/GB2022/050792

§ 371 Date: Oct. 2, 2023

(30) Foreign Application Priority Data
Mar. 30, 2021 (GB) 2104539.8

(51) Int. Cl. G16H 50/30; A61B 5/00

(52) U.S. Cl. CPC G16H 50/30; A61B 5/4064

(57) ABSTRACT

Systems, methods, computer program products, and computer readable media for predicting brain biomarker status are described. Techniques are described for receiving response data comprising a plurality of inputs by a subject and at least one input characteristic associated with the plurality of inputs, receiving characteristic data comprising a parameter indicative of a characteristic associated with the subject, processing the response data and the characteristic data, comparing the processed response data to reference response data or to model reference data, comparing the processed characteristic data to corresponding reference characteristic data or to model characteristic data, and determining a status of at least one biomarker of a brain disorder in the brain of the subject based on the comparisons.

### FIELD OF THE INVENTION

The present technology relates to systems, methods, computer program products, and computer readable media for predicting brain biomarker status. In particular, the technology relates to systems, methods, computer program products and computer readable media for providing an indication of the status of a biomarker of a brain disorder in the brain of a candidate subject.

### BACKGROUND

Disorders of the brain affect many people around the globe and such disorders come in various different forms. Neurological disorders may include but not be limited to mental disorders, neuropsychiatric disorders, neurodegenerative disorders and neuromuscular disorders. Some of these disorders may be manageable in day-to-day life. Others may be life-threatening or terminal. In order to achieve better outcomes or to provide more suitable treatment and care, it would be beneficial to be able to more accurately identify patients that have an increased risk of developing a brain disorder and/or to be able to identify the development of a brain disorder at an early stage. Further, following identification of at-risk patients or patients with a brain disorder, it is desirable to be able to accurately screen, stratify and treat patients in order to improve care and treatment to yield better outcomes and/or to provide more suitable care. In other words, outcomes can be improved, and treatments can be optimised if the risk of developing such disorders is able to be determined and/or if such disorders are able to be diagnosed at an early stage of development.

### SUMMARY

Examples or preferred aspects and embodiments of the present technology are set out in the accompanying independent and dependent claims.

This Summary is intended to introduce a selection of concepts in a simplified form that are further described below in the Detailed Description. This Summary is not intended to identify key features or essential features of the claimed subject-matter, nor is it intended to be used to limit the scope of the claimed subject-matter.

According to a first aspect of the present technology, there is provided a method for predicting brain biomarker status. The method comprises receiving at least one set of response data comprising a plurality of inputs by a subject and at least one input characteristic, wherein each of the inputs comprises a determination as to whether or not each image of a series of natural test images displayed satisfies at least one predetermined categorisation criterion, and wherein each input characteristic comprises data representative of at least one feature associated with one or more of the inputs; receiving at least one set of characteristic data comprising at least one parameter indicative of a characteristic associated with the subject; extracting the at least one input characteristic from the response data to generate at least one set of processed response data; processing the at least one set of characteristic data; performing a first comparison by comparing the at least one set of processed response data to corresponding reference response data or to corresponding model reference response data derived from reference response data, wherein the reference response data or the model reference response data is indicative of a status of at least one biomarker of a brain disorder; performing a second comparison by comparing the at least one set of processed characteristic data to corresponding reference characteristic data or to corresponding model characteristic data derived from reference characteristic data wherein the reference characteristic data or the model characteristic data is indicative of the status of the at least one biomarker of the brain disorder; and determining the status of the at least one biomarker of the brain disorder in the brain of the subject based on the first comparison and the second comparison.

In this way, a brain biomarker status of a candidate subject can be predicted. A brain biomarker status may be indicative of an increased, or decreased, risk of developing a brain disorder. A brain biomarker status may be indicative of the presence or absence of a brain disorder. A brain biomarker status may be indicative the stage of the brain disorder (e.g. early onset).

In some preferred examples, the at least one set of response data comprises a plurality of sets of response data each obtained at a different respective time point. As such, additional data relating to a given candidate subject is received, such that a more comprehensive dataset for a given candidate is available.

In some preferred examples, each different respective time point is separated by a predetermined time interval.

In some preferred examples, the method comprises performing a third comparison by comparing each set of processed response data to each of the other respective sets of processed response data; and determining a change in response data over time based on the third comparison, wherein determining the status of the at least one biomarker of the brain disorder is further based on the determined change in response data. In this way, the brain biomarker status can be estimated based on an additional intra-subject comparison.

In some preferred examples, the status of the at least one biomarker of the brain disorder comprises at least one of biomarker positivity, concentration, volume or size of the at least one biomarker in the brain of the subject.

In some preferred examples, the method comprises determining that the status of the at least one biomarker of the brain disorder exceeds a predetermined threshold. In this way, a determination as to the relative state of the brain biomarker status is enabled.

In some preferred examples, the method comprises determining that the status of the at least one biomarker of the brain disorder is lower than a predetermined threshold. In this way, a determination as to the relative state of the brain biomarker status is enabled.

In some preferred examples, the method comprises determining an indication of a risk of the subject developing at least one brain disorder based on the determined status of the at least one biomarker. In this way, the risk of the subject of developing a brain disorder is determined, thereby allowing the candidate subject to potentially take preventative measures to lower the risk.

In some preferred examples, the method comprises detecting an indication of the presence of at least one brain disorder based on the determined status of the at least one biomarker. In other examples, the method may further comprise determining at least one therapeutic to be administered based on the detected indication of the at least one brain disorder. In still further examples, the method further comprises determining at least one non-pharmacological treatment to be implemented based on the detected indication of the at least one brain disorder. This allows a candidate subject and/or clinical staff to make educated decisions on treatment and care, enabling more suitable treatments to be recommended to the candidate subject.

In some preferred examples, the brain disorder is at least one of Alzheimer's Disease, cerebrovascular dementia, mild cognitive impairment, frontotemporal dementia, dementia with Lewy Bodies, multiple sclerosis, motor neurone disease, Parkinson's disease, attention deficit hyperactivity disorder, primary mental illness Huntington's disease, depression, post-traumatic stress disorder or brain injury.

In some preferred examples, the at least one biomarker comprises at least one of neurofilament light chain, amyloid beta or Tau.

In some preferred examples, the method further comprises adding the at least one set of response data to the reference response data and the at least one set of characteristic data to the reference characteristic data once the biomarker status of the subject has been obtained. In this way, the reference datasets can be updated, resulting in a dynamic method for estimating brain biomarker status.

In some preferred examples, the reference characteristic data comprises a first set of characteristic data pre-obtained from a plurality of subjects each with at least one brain disorder and a second set of characteristic data pre-obtained from a plurality of control subjects, and the reference response data comprises a first set of response data pre-obtained from a plurality of subjects each with at least one brain disorder and a second set of response data pre-obtained from a plurality of control subjects. In this way, the candidate data can be compared to data acquired from healthy and non-healthy cohorts, resulting in a more accurate estimation of the brain health and biomarker status of the candidate subject.

In some preferred examples, the characteristic data comprises at least one of personal data, medical data, medical history data, family medical history data, lifestyle data, clinical data, or genetic data.

In some preferred examples, the predetermined categorisation criterion is whether the image includes an animal.

In some preferred examples, the at least one input characteristic associated with the plurality of inputs comprises at least one of: individual response times for each of the plurality of inputs, a cumulative response time for the plurality of inputs, a mean response time, individual accuracy scores for each of the plurality of inputs, a cumulative accuracy score for the plurality of inputs or an overall accuracy score of the plurality of inputs. In this way, various measures associated with the input can be processed and used in the first comparison.

In some preferred examples, the method comprises determining a speed score of the plurality of inputs of the subject based on the input characteristic, wherein the input characteristic comprises the individual response times and the individual response times are within a predetermined range. This allows a further parameter to be deduced for the candidate subject to yield a more comprehensive representation of the performance of the candidate in the task.

In some preferred examples, the method comprises determining a test index based on the overall accuracy score and the speed score. In a further example, the method comprises calculating a plurality of predetermined statistical values of the test index and determining an attention score based on the test index and the calculated statistical values of the test index. This allows a further parameter to be deduced for the candidate subject to yield a more comprehensive representation of the performance of the candidate in the task.

According to a second aspect of the present technology, there is provided a system for predicting brain biomarker status. The system comprises at least one processor and memory storing computer-executable instructions that, when executed by the one or more processors, cause the at least one processor to receive at least one set of response data comprising a plurality of inputs by a subject and at least one input characteristic comprising data representative of at least one feature associated with one or more of the inputs, wherein each of the inputs comprises a determination as to whether or not each image of a series of natural test images displayed satisfies at least one predetermined categorisation criterion; receive at least one set of characteristic data comprising at least one parameter indicative of a characteristic associated with the subject; extract the at least one input characteristic from the response data to generate at least one set of processed response data; process the at least one set of characteristic data; perform a first comparison by comparing the at least one set of processed response data to corresponding reference response data or to corresponding model reference response data derived from reference response data, wherein the reference response data or the model reference response data is indicative of a status of at least one biomarker of a brain disorder; perform a second comparison by comparing the at least one set of processed characteristic data to corresponding reference characteristic data or to corresponding model characteristic data derived from reference characteristic data, wherein the reference characteristic data or the model characteristic data is indicative of the status of the at least one biomarker of the brain disorder; and determine the status of the at least one biomarker of the brain disorder in the brain of the subject based on the first comparison and the second comparison.

In this way, a brain biomarker status of a candidate subject can be predicted using the system.

In a third aspect of the present technology, there is provided a computer program product comprising instructions which, when the program is executed by a computer, cause the computer to receive at least one set of response data comprising a plurality of inputs by a subject and at least one input characteristic, wherein each of the inputs comprises a determination as to whether or not each image of a series of natural test images displayed satisfies at least one predetermined categorisation criterion, and wherein each input characteristic comprises data representative of at least one feature associated with one or more of the inputs; receive at least one set of characteristic data comprising at least one parameter indicative of a characteristic associated with the subject; extract the at least one input characteristic from the response data to generate at least one set of processed response data; process the at least one set of characteristic data; perform a first comparison by comparing the at least one set of processed response data to corresponding reference response data or to corresponding model reference response data derived from reference response data, wherein the reference response data or the model reference response data is indicative of a status of at least one biomarker of a brain disorder; perform a second comparison by comparing the at least one set of processed characteristic data to corresponding reference characteristic data or to corresponding model characteristic data derived from reference characteristic data wherein the reference characteristic data or the model characteristic data is indicative of the status of the at least one biomarker of the brain disorder; and determine the status of the at least one biomarker of the brain disorder in the brain of the subject based on the first comparison and the second comparison.

In this way, a brain biomarker status of a candidate subject can be predicted using the computer program product.

In a fourth aspect of the present technology, there is provided a computer-readable medium comprising instructions which, when executed by a computer, cause the computer to receive at least one set of response data comprising a plurality of inputs by a subject and at least one input characteristic, wherein each of the inputs comprises a determination as to whether or not each image of a series of natural test images displayed satisfies at least one predetermined categorisation criterion and wherein each input characteristic comprises data representative of at least one feature associated with one or more of the inputs; receive at least one set of characteristic data comprising at least one parameter indicative of a characteristic associated with the subject; extract the at least one input characteristic from the response fata to generate at least one set of processed response data; process the at least one set of characteristic data; perform a first comparison by comparing the at least one set of processed response data to corresponding reference response data or to corresponding model reference data derived from reference response data, wherein the reference response data or the model reference response data is indicative of a status of at least one biomarker of a brain disorder; perform a second comparison by comparing the at least one set of processed characteristic data to corresponding reference characteristic data or to corresponding model characteristic data derived from reference characteristic data, wherein the reference characteristic data or the model characteristic data is indicative of the status of the at least one biomarker of the brain disorder; and determine the status of the at least one biomarker of the brain disorder in the brain of the subject based on the first comparison and the second comparison.

In this way, a brain biomarker status of a candidate subject can be predicted using the computer-readable medium.

In a fifth aspect of the present technology, there is provided a method of treatment for pharmacologically treating a brain disorder. The method comprises receiving at least one set of response data comprising a plurality of inputs by a subject and at least one input characteristic, wherein each of the inputs comprises a determination as to whether or not each image of a series of natural test images displayed satisfies at least one predetermined categorisation criterion, and wherein each input characteristic comprises data representative of at least one feature associated with one or more of the inputs; receiving at least one set of characteristic data comprising at least one parameter indicative of a characteristic associated with the subject; extracting the at least one input characteristic from the response data to generate at least one set of processed response data; processing the at least one set of characteristic data; performing a first comparison by comparing the at least one set of processed response data to corresponding reference response data or to corresponding model reference data derived from reference response data, wherein the reference response data or the model reference response data is indicative of a status of at least one biomarker of a brain disorder; performing a second comparison by comparing the at least one set of processed characteristic data to corresponding reference characteristic data or to corresponding model characteristic data derived from reference characteristic data wherein the reference characteristic data or the model characteristic data is indicative of the status of the at least one biomarker of the brain disorder; determining the status of the at least one biomarker of the brain disorder in the brain of the subject based on the first comparison and the second comparison; detecting an indication of the presence of at least one brain disorder based on the determined status of the at least one biomarker; and determining at least one therapeutic to be administered based on the detected indication of the at least one brain disorder; and administering said at least one therapeutic. In this way, pharmacological treatments are administrable based on the detected indication of the presence of a brain disorder.

In a sixth aspect of the present technology, there is provided a method of obtaining an indication of the efficacy of the therapeutic administered according to the fifth aspect. The method comprises performing the method of the first aspect at a first time point; administering a therapeutic according to the fifth aspect; performing the method of the first aspect at a second time point following the administration of the therapeutic; performing a comparison of the status of the at least one biomarker of the brain disorder at the first time point to the status of the at least one biomarker of the brain disorder at the second time point; and determining, based on the comparison, the efficacy of the at least one therapeutic. In this way, the efficacy of the therapeutic administered in response to the detected indication of the brain disorder is assessed.

According to a seventh aspect of the present technology, there is provided a method of treatment for treating a brain disorder with non-pharmacological means. The method comprises receiving at least one set of response data comprising a plurality of inputs by a subject and at least one input characteristic, wherein each of the inputs comprises a determination as to whether or not each image of a series of natural test images displayed satisfies at least one predetermined categorisation criterion, and wherein each input characteristic comprises data representative of at least one feature associated with one or more of the inputs; receiving at least one set of characteristic data comprising at least one parameter indicative of a characteristic associated with the subject; extracting the at least one input characteristic from the response data to generate at least one set of processed response data; processing the at least one set of characteristic data; performing a first comparison by comparing the at least one set of processed response data to corresponding reference response data or to corresponding model reference data derived from reference response data, wherein the reference response data or the model reference response data is indicative of a status of at least one biomarker of a brain disorder; performing a second comparison by comparing the at least one set of processed characteristic data to corresponding reference characteristic data or to corresponding model characteristic data derived from reference characteristic data, wherein the reference characteristic data or the model characteristic data is indicative of the status of the at least one biomarker of the brain disorder; determining the status of the at least one biomarker of the brain disorder in the brain of the subject based on the first comparison and the second comparison; detecting an indication of the presence of at least one brain disorder based on the determined status of the at least one biomarker; and determining at least one non-pharmacological treatment to be implemented based on the detected indication of the at least one brain disorder. In this way, non-pharmacological treatments may be employed or utilised based on the detected indication of the presence of a brain disorder.

In an eighth aspect of the present technology, there is provided a method of obtaining an indication of the efficacy of the non-pharmacological treatment to be implemented according to the seventh aspect. The method comprises performing the method of the first aspect at a first time point; administering a therapeutic according to the seventh aspect; performing the method of the first aspect at a second time point following the implementation of the non-pharmacological treatment; performing a comparison of the status of the at least one biomarker of the brain disorder at the first time point to the status of the at least one biomarker of the brain disorder at the second time point; and determining, based on the comparison, the efficacy of the at least one non-pharmacological treatment. In this way, the efficacy of the non-pharmacological therapeutic administered in response to the detected indication of the brain disorder is assessed.

According to a ninth aspect of the present technology, there is provided a method for screening subjects. The method comprises performing the method according to the first aspect for a plurality of candidate subjects; performing, for each of the plurality of subjects, a comparison of the status of the at least one biomarker of the brain disorder to the corresponding status of the at least one biomarker of the brain disorder in at least one other subject; and assessing the risk of each subject of developing the at least one brain disorder based on each comparison.

In this way, an improved indication of the relative risk of a brain disorder is determined, allowing subjects to be more effectively screened for potential treatment, care and/or for further research.

According to a tenth aspect of the present technology, there is provided a method for subject stratification. The method comprises performing the method according to the first aspect for a plurality of candidate subjects; performing, for each subject, a comparison of the status of the at least one biomarker of the brain disorder to the corresponding status of the at least one biomarker of the brain disorder in at least one other subject; and classifying, based on the comparison, each of the plurality of subjects into a plurality of sub-groups.

According to an eleventh aspect of the present technology, there is provided a method for determining a suitability of a treatment for a given subject. The method comprises performing the method according to the first aspect for the given subject; and determining, based on the determined status of the at least one biomarker, the suitability of the treatment for the given candidate subject.

In this way, more similar candidate subjects in terms of brain biomarker status are able to be grouped together.

It will be apparent to anyone of ordinary skill in the art, that some of the features indicated above as preferable in the context of one of the aspects of the present technology may replace one or more of the preferred features of other preferred features of the present technology. Such apparent combinations are not explicitly listed above under each such possible additional aspect for the sake of conciseness.

Other examples will become apparent for the following detailed description, which, when taken in conjunction with the drawings, illustrate by way of example the principles of the present technology.

### BRIEF DESCRIPTION OF THE DRAWINGS

The accompanying drawings illustrate various examples of the present technology. Common reference numerals are used throughout the figures, where appropriate, to indicate similar features.

FIG. 1 illustrates an exemplary procedure for a computerised image categorisation task.

FIG. 2 depicts an exemplary method for collecting and analysing training data.

FIG. 3 illustrates the process for calculating a vector of image statistics.

FIG. 4 illustrates an exemplary method for predicting brain biomarker status.

FIG. 5 is a schematic illustration of a system for predicting a status of brain biomarkers.

FIG. 6 illustrates an exemplary method of pharmacological treatment according to aspects of the present invention.

FIG. 7 illustrates an exemplary method of non-pharmacological treatment according to aspects of the present invention.

FIG. 8 illustrates an exemplary method of screening potential patients.

FIG. 9 illustrates an exemplary method of patient stratification.

FIG. 10 illustrates an exemplary method for determining a suitability of a treatment for a given subject.

The accompanying drawings illustrate various examples. Common reference numerals are used throughout the figures, where appropriate, to indicate similar features.

### DETAILED DESCRIPTION

The following description is made for the purpose of illustrating the general principles of the present technology and not intended to limit the inventive concepts as outlined herein. The following description is presented by way of example to enable a person skilled in the art to make and use the present technology. The present technology is not limited to the embodiments described herein and various modifications to the disclosed embodiments will be readily apparent to anyone skilled in the art.

The present technology relates to systems, methods, computer program products, and computer readable media for predicting brain biomarker status. In particular, the technology relates to systems and methods for providing an indication of the status of a biomarker of a brain disorder in the brain of a candidate subject.

Brain disorders are common globally and are present in many different forms. Neurological disorders may include but not be limited to mental disorders, neuropsychiatric disorders, neurodegenerative disorders and neuromuscular disorders.

As an example, neurodegenerative disorders include Alzheimer's disease. The Alzheimer's Association estimates that 60 to 80% of cases of dementia are attributable to Alzheimer's disease; dementia is a syndrome which is associated with a progressive decline in memory and cognitive ability. Mild cognitive impairment (MCI) is a pre-dementia condition that has a prevalence ranging between 16 to 20% in a population of 60 to 89-year olds (Sachdev, P. S. et al. (2015) ‘The prevalence of mild cognitive impairment in diverse geographical and ethnocultural regions: The COSMIC Collaboration.’ PLOS One 10(11), pp. 1-19). Of individuals with MCI, it is estimated that 5 to 15% of them develop dementia every year (Petersen, R. C. et al. (2018) ‘Practice guideline update summary: Mild cognitive impairment’, Neurology, 90(3), pp. 126-135).

Further, following a diagnosis of Alzheimer's disease, the average life expectancy of the patient is approximately ten years. Whilst the rate of deterioration differs amongst individuals, the progressive nature of the disease means that many patients experience a decline in their quality of life over time. Currently, there is no cure for Alzheimer's disease, but an array of treatments are available in order to potentially relieve some of the symptoms.

As such, due to the relative prevalence and personal impact of such disorders, there is a clear need to be able to more accurately determine an increased risk of developing a brain disorder and/or to be able to identify brain disorders at an early stage. Owing to the progressive nature of many of these disorders, they can be particularly challenging to diagnose in the early stages. This is problematic as diagnosing such disorders at a late stage can result in poorer clinical outcomes for the patient. This, coupled with the social and economic impact of late-stage diagnoses, can be devastating for patient outcomes.

Further, timely identification of brain disorders is desirable in order to provide a formal diagnosis, enabling suitable treatments to be determined and administered, or to enable suitable care to be provided. This is beneficial for the patient, family and caregivers, as well as their healthcare provider and wider society (Dubois B. et al. (2015) ‘Timely diagnosis for Alzheimer's disease: A literature review on benefits and challenges’, Journal of Alzheimer's Disease, 49(3), pp. 617-631). Crucially, there is evidence to suggest that early detection can allow preventative or protective measures to be implemented in order to slow or limit the progression of the brain disorder (Sabbagh, M. N. et al. (2020) ‘Rationale for Early Diagnosis of Mild Cognitive Impairment (MCI) supported by Emerging Digital Technologies’, The Journal of Prevention of Alzheimer's Disease, 7, pp. 1-7; Clare, L. et al. (2017) ‘Potentially modifiable lifestyle factors, cognitive reserve, and cognitive function in later life: A cross-sectional study’, PLOS Medicine, 14(3), pp. 1-14).

Typically, for a given brain disorder, there is no single diagnostic test that can provide a definitive identification of the disorder. This is true in the case of Alzheimer's disease. Alzheimer's disease may be diagnosed based on cognitive or neuropsychological assessments, brain scans and/or on the basis of further qualitative information that may describe how well the patient is coping with day-to-day activities, along with any corresponding long- or short- term changes in the ability of the patient to manage everyday activities. As such, cognitive and/or neuropsychological assessments are key in obtaining a formal diagnosis of Alzheimer's disease. This is also the case for many other brain disorders.

Computerised cognitive tests have been developed to aid in the early detection of brain disorders. These tests primarily have been designed to mirror traditional cognitive assessments such that they are typically not designed to acquire additional potentially diagnostically useful cognitive information (Parsey, C. M. and Schmitter-Edgecombe, M. (2013) ‘Applications of Technology in Neuropsychological Assessment’, The Clinical Neuropsychologist, 27(8), pp. 1328-1361). Additionally, computerised cognitive assessments may be considered to lack specificity and sensitivity and thus yield less accurate results than conventional neuropsychological testing (Koo, B. M. and Vizer, L. M. (2019) ‘Mobile Technology for Cognitive Assessment of Older Adults: A Scoping Review’, Innovation in Aging, 3(1), pp. 1-14). There is a need to provide a more accessible and rapid neuropsychological testing tool that can yield more accurate and comprehensive results for clinical use, potentially for aiding in the early diagnosis of brain disorders.

As described previously, brain disorders can be challenging to diagnose. However, indications of the risk, presence, prognosis or severity of a brain disorder may be inferred from the status of a brain biomarker. Generally, biomarkers are considered to be measurable biological attributes within a subject and they may be representative of a particular feature in a subject or they may be indicative of the state or condition of at least one disorder in a subject. Biomarkers may encompass “a defined characteristic that is measured as an indicator of normal biological processes, pathogenic processes, or responses to an exposure of intervention, including therapeutic interventions” (FDA-NIH Biomarker Working Group. (2016) ‘BEST (Biomarkers, EndpointS, and other Tools) Resource’. Silver Spring (MD): Food and Drug Administration (US)). Thus, brain biomarkers may comprise: a defined characteristic that is measured as an indicator of normal biological processes that occur in or affect the brain; pathogenic processes that occur in or affect the brain; or responses to an exposure of intervention, including therapeutic interventions, whereby the intervention occurs in or affects the brain.

Brain biomarkers may also be biomarkers that may be representative of or descriptive of processes in a brain. Brain biomarkers may comprise neuroimaging markers, fluid biomarkers, biochemical markers, or other structural or functional markers in the brain. The status of these brain biomarkers may enable a determination as to the risk of a subject developing a given brain disorder. The status of these brain biomarkers may be indicative of the presence of a brain disorder, or of the prognosis of a patient with formal diagnosis of a brain disorder.

Status of a brain biomarker may be expressed as brain biomarker positivity. Brain biomarker positivity may be associated with Alzheimer's disease or other brain disorders. Positivity of cerebrospinal amyloid-beta₄₂ (CSF Aβ₄₂) may be assessed using an enzyme-linked immunosorbent assay (ELISA) or a Luminex assay, and ranges for CSF Aβ₄₂ positivity may differ accordingly. For example, where CSF Aβ₄₂ is measured using ELISA, the cut-off for CSF Aβ₄₂ positivity may range from 450-650 pg/mL. Where CSF Aβ₄₂ is measured using a Luminex assay, the cut-off for CSF Aβ₄₂ positivity is considered to be 192 pg/mL. Further, through the employment of positron emission tomography (PET), the positivity of amyloid PET may be assessed and different ligands including a ¹¹C-labeled modified derivative of amyloid-binding Thioflavin T known as Pittsburgh Compound-B (PiB), flutemetamol or florbetapir may be used. The method of evaluation may differ for each of these ligands. For example, the method of evaluation when using the PiB ligand may be through the usage of the mean cortical binding potential, the global cortical normalised binding potential, the region-of-interest PiB retention, the mean cortical PiB retention, the mean cortical SUVR or the global cortical PiB retention. The amyloid PET positivity cut-offs for each of these respective methods of evaluation has been shown to be approximately less than 0.2, 0.5, 1.6, 1.6, 1.465 and 1.11. When using flutemetamol, the method of evaluation may be the mean cortical SUVR, with a cut-off of less than 1.42. When using florbetapir, the method of evaluation may be mean cortical retention, with a cut-off of less than 1.11 (Blennow, K. et al. (2015) ‘Amyloid biomarkers in Alzheimer's disease’, Trends in Pharmacological Science, 36(5), pp 297-209).

A status of a brain biomarker may be expressed purely as a concentration. One study showed significantly lower levels of cerebrospinal fluid amyloid beta₁₋₄₂ in Alzheimer's patients in comparison to healthy controls (183 pg/mL vs. 491 pg/mL respectively). The same study showed significantly increased levels of CSF Tau in Alzheimer's patients in comparison to healthy controls (587 pg/mL vs. 224 pg/mL respectively)(Sunderland, T. et al. (2003) ‘Decreased β-Amyloid₁₋₄₂ and Increased Tau Levels in Cerebrospinal Fluid of Patients With Alzheimer's Disease’, Journal of the American Medical Association, 289(16), pp. 2094-2103).

It is clear that the relative status of a given brain biomarker may be a potentially clinically useful tool in relation to brain health. However, at present, the known brain biomarkers of brain disorders are not easily accessible or scalable, such that they are not routinely available (Ritchie, C. W. et al. (2017) ‘The Edinburgh Consensus: preparing for the advent of disease-modifying therapies for Alzheimer's disease’, Alzheimer's Research & Therapy, 9(1), pp. 1-7). It is therefore desirable to provide systems and methods that can accurately and easily provide estimations of brain biomarker status. This would aid in risk determination and diagnosis and improve the ability to monitor disease progression. This would in turn enable improvements in the ability to screen, stratify and treat patients, and to enable the efficacy of treatments to be assessed. Further, biomarker status may provide indications as to the suitability of treatments for given subjects.

The present technology relates to systems, methods, computer program products, and computer readable media for predicting brain biomarker status. In particular, the technology relates to systems and methods for providing an indication of the status of a biomarker of a brain disorder in the brain of a candidate subject.

The status of the biomarker in the brain may be indicative of the risk of developing at least one brain disorder. The status of the biomarker in the brain may enable an indication of the presence of at least one brain disorder to be determined. The status of the biomarker in the brain may enable a determination as to the prognosis of a previously diagnosed brain disorder. The status of the biomarker in the brain may provide an indication as to the severity of a brain disorder that is present in the subject.

The term “brain disorder(s)” will hereinafter be used to encompass brain disorders and impairments of the brain that may relate to precursors of brain disorder such as mild cognitive impairment. The brain disorder may include at least one of Alzheimer's Disease, cerebrovascular dementia, mild cognitive impairment, frontotemporal dementia, dementia with Lewy Bodies, multiple sclerosis, motor neurone disease, Parkinson's disease, attention deficit hyperactivity disorder, primary mental illness Huntington's disease, depression, post-traumatic stress disorder or brain injury.

The present technology adopts a similar categorisation task as to the task outlined previously in WO2015067945A1. WO2015067945A1 relates to a system for assessing a mental health disorder. A rapid categorisation task is provided to a subject, presenting the subject with a series of natural test images. The categorisation task requires the subject to input a response, following the display of a given test image, as to whether or not the test images satisfies a predetermined categorisation criterion. The response time of the subject is recorded. In essence, a plurality of sets of data for a candidate subject are received, with a first set relating to the results of a categorisation task (e.g., whether the response was correct or incorrect) and the second set relating to data associated with the subject (e.g., the relative response time of the subject). The plurality of data sets are processed and compared to reference data in order to assess whether the subject has, or is likely to develop, a mental health disorder.

In the present technology, the sets of candidate data are compared to a corresponding set of reference data, wherein the reference data has been previously mapped to a brain biomarker status. The reference data may comprise actual measured values, predicted values or a combination of both. Based on these comparisons, a status of a brain biomarker of a brain disorder in the brain of the candidate subject is determined.

The categorisation task is designed to test various different aspects of brain functionality, as opposed to merely one aspect of brain functionality such as memory impairment. This allows a more detailed and complex analysis of the brain functionality of the subject to be deduced. As a consequence, underlying signs and mild symptoms that may not yet be noticeable to the subject may be identified, allowing early diagnosis of a brain disorder or identification of an increased risk of developing a brain disorder. This allows measures to be taken to reduce the impact or to slow the onset of the disorder. Further, it may enable preventative measures to be taken.

In the present technology, a human subject may perform a computerised image categorisation task at a given time point. The subject may be any human individual. The subject may be a healthy subject, in which the subject is considered to not have any brain disorders. The subject may be a non-healthy subject in which the subject is considered to have a brain disorder. The basic methodology for the task is the same whether the subject is a training subject or a candidate subject.

During the categorisation task, the subject is presented with a series of natural images. The series of natural images comprise a plurality of natural images. The term “natural images” refers to images, including but not limited to photographs and drawings of natural scenes. In other words, natural images relate to images of recognisable scenes that the human visual system is accustomed to seeing, as opposed to more abstract visual representations, images, or drawings of scenes. In the present technology, natural images are comprised of images having a similar statistical structure to images that the human visual system is accustomed to viewing. Examples of such images may include photographs of scenes depicting the natural world e.g. rivers, mountains or forests or man-made aspects of modern life e.g. streets, buildings, or car parks. Whilst the latter are “man-made”, they are considered to constitute “natural scenes” in this context as they are readily recognisable to a healthy observer; i.e. they are readily distinguished from abstract, two-dimensional representations of such scenes that may not be immediately recognisable by the viewer. As such, these “natural images” may be interchangeably described as “everyday images” or “recognisable images”.

Upon presentation of each of the images in the series, the subject is tasked with assigning each of the natural images to a predefined category. In an example, the subject may have to determine whether the presented image includes a given feature or object. By way of example, the image may contain an elephant and the subject is expected to indicate whether or not an animal is present; in such a case, the correct assignment would be “YES”. In another example, the subject may have to determine whether or not a given feature is as specified; by way of example, the subject may be expected to indicate whether or not a feature in the image is an elephant.

The one of ordinary skill in the art appreciates that as each of the images in the series is distinct, a given categorisation task is not limited to categorisation according to a given feature or according to the presence or absence of the feature; both of these assignment categories may be utilised in the same categorisation task.

As described above, categorisation of each natural image in the series requires a separate input or response from the subject; the terms “input” and “response” in this context may be used interchangeably. Each of the inputs are detected. Upon completion or termination of the task, the response data is collated, the response data including each of the detected inputs. It is apparent to one of ordinary skill in the art that should the task be terminated early, or incomplete, that a set of response data may still be collated and used for subsequent analysis, provided a plurality of responses from the subject are available.

The objective of the categorisation task is to correctly categorise the natural images as quickly as possible. In addition to the inputs, during the task, at least one input characteristic is detected. The input characteristic is a characteristic or feature associated with the inputs. Optionally, the output of the categorisation task may be analysed in isolation in order to detect indications of symptoms of brain disorders or early-onset disorders. For example, the analysis of the response data may reveal memory loss, or indicate that the subject has difficulties in concentrating, or exhibit signs of confusion or prosopagnosia, particularly as individuals displaying such symptoms commonly may have difficulties in identifying objects in images.

It is known that reaction times may be affected by different types of brain disorders (Gordon, B. and Carson, K. (1990) ‘The basis for choice reaction time slowing in Alzheimer's disease’, Brain and Cognition, 13(2), pp. 148-166; Jahanshahi, M. et al. (1992) ‘Simple and Choice Reaction Time and the Use of Advance Information for Motor Preparation in Parkinson's Disease’, Brain, 115(2), pp. 539-564; Knopman, D. and Nissen, M. J. (1991) ‘Procedural learning is impaired in Huntington's disease: Evidence from the serial reaction time task’, Neuropsychologia, 29(3), pp. 245-254; Rinehart, N. J. et al (2001) ‘Movement Preparation in High-Functioning Autism and Asperger Disorder: A Serial Choice Reaction Time Task Involving Motor Reprogramming’, 31(1), pp. 79-88). As such, the speed of the response of the subject in the categorisation task may be useful for inferring the presence of a brain disorder. Further, it has been shown that the reaction times of healthy subjects (i.e. those without any evidence of a precursor to a brain disorder or a brain disorder proper) are correlated with statistical properties of natural images (Mirzaei, A. et al. (2013) ‘Predicting the Human Reaction Time Based On Natural Image Statistics in a Rapid Categorization Task’, 81, pp. 36-44). As a consequence, it is expected to see different patterns of correlation between reaction times and statistical properties of natural images in healthy subjects and subjects with brain disorders. It is expected to see a noticeable difference between healthy subjects and those with early onset symptoms and potentially more substantial differences between healthy subjects and those in which a brain disorder is more advanced.

As described above, the input characteristic is a characteristic or feature associated with the inputs and is therefore representative of a given input. In a first example, the input characteristic is a feature that is associated with each individual input. The input characteristic may comprise at least one of individual response times for each input, or individual accuracy scores for each input. In a second example, the input characteristic is a feature that is associated with a subset of the plurality of inputs. The input characteristic may comprise a cumulative response time for the subset of the plurality of inputs, a mean response time for the subset of the plurality of inputs, a cumulative accuracy score for the subset of the plurality of inputs or overall accuracy score for the subset of the plurality of inputs. In a third example, the input characteristic is a feature that is associated with the totality of inputs. The input characteristic may comprise a cumulative response time for all of the inputs, a mean response time for all of the inputs, a cumulative accuracy score over all of the inputs or a mean accuracy score over all of the inputs.

The mean accuracy score Au is described according to:

$$A μ = \frac{Number of correct categorisations}{Total number of images} \times 100 \qquad ( 1 )$$

Optionally, additional parameters that are further representative of the inputs may be derived. In some examples, these additional parameters are derivable solely from the input characteristic data. The additional parameters may comprise a speed score, a test index, an accuracy performance slope, a speed performance slope, a test index performance slope, an accuracy score per subcategory of images, a speed score per subcategory of images, a test index score per subcategory of images, an attention score, a consecutive accuracy score or a consecutive inaccuracy score.

In an example, a speed score for each of the plurality of inputs is determined based on the input characteristic, wherein the input characteristic comprises the individual response times within a predetermined range. In other words, individual response times that are considered to be outliers in accordance with the boxplot method are removed. A speed score S is calculable according to:

$$S = min [ 100 , 100 \times {e}^{\frac{- mean correct RT}{1025} + 0.341} ] \qquad ( 2 )$$

where:
RT is response time; and
e is Euler's number.

In additional or alternative examples, a test index may be determined. The test index TI is described by:

$$TI = ( \frac{A μ}{100} \times \frac{Speed}{100} ) \times 100 \qquad ( 3 )$$

where:
Aμ is the mean accuracy score calculated according to equation (1); and
S is the speed score calculated according to equation (2).

In additional or alternative examples, an accuracy performance slope, a speed performance slope or a test index performance slope may be determined. Whilst being separate metrics, each of the performance slopes are determined in a similar manner and therefore are discussed together for brevity. To calculate the performance slopes, the test images are divided into ten bins of equal size. Within each bin, the relevant metric is calculated according to equations (1), (2) or (3) depending on which performance slope is being determined. For each calculated metric, the slope coefficient α is calculated using the least squares method for linear fit according to:

$$α = \frac{{\sum }_{i = 1}^{n} ( {x}_{i} - \underline{X} ) ( {y}_{i} - \underline{Y} )}{{\sum }_{i = 1}^{n} {( {x}_{i} - \underline{X} )}^{2}} \qquad ( 4 )$$

where:
x is the bin numerical value of the bin;
y is the metric variable;
n is the number of bins;
X is the mean of x values; and
Y is the mean of y values.

In additional or alternative examples, the overall accuracy, speed and test index scores may be calculated per subcategory of images. Images within the series are ranked according to their difficulty level. The difficulty level is determined based on the accuracy of responses previously received from healthy subjects for these images. Within each of the four categories, the overall accuracy, speed and test index scores are calculable according to respective equations (1), (2) and (3).

In additional or alternative examples, an attention score may be calculated. The attention score relates to the relative level of focus of the subject throughout the duration of the task. The performance of the subject throughout the task is continuously monitored. Fluctuations in the performance level of the subject above a predetermined threshold may be indicative of a subject not engaging with the task. Such a fluctuation may be due to the subject generally having difficulties with concentration. In contrast, a single fluctuation in performance above the predetermined threshold during the task may be indicative of an isolated event during the task that has caused the subject to be inadvertently distracted.

In additional or alternative examples, a consecutive accuracy score may be determined. The consecutive accuracy score is descriptive of the number of consecutive responses that classify the image correctly. By way of example, the consecutive accuracy score comprises the length of the longest chain of consecutive correct responses. Similarly, a consecutive inaccuracy score may be determined. The consecutive inaccuracy score is descriptive of the number of consecutive responses that classify the image incorrectly. By way of example, the consecutive inaccuracy score comprises the length of the longest chain of consecutive incorrect responses.

The categorisation task may be completed at a plurality of time points. The categorisation task completed at additional time points need not be identical to the first task completed i.e. a different series of images may be displayed. It is appreciated that the results of the first task and additional tasks may be compared to reveal the progression of symptoms or of the brain disorder or alternatively, to track improvements in brain health following the onset of treatment for the detected disorder or symptoms. It is appreciated that the subject may complete any number of additional tasks at given time intervals, depending on the nature of the brain health of the subject and the personal circumstances of the subject. It is appreciated that data received at additional time points for training subjects may also be included in the training set.

During each task, a series of individual greyscale natural images acting as a stimulus are displayed on a computer screen. Each image is displayed for 100±50 ms. In an example, each natural image comprises an animal or a non-animal, wherein the non-animal image portrays an image of a scene that does not include an animal. In this example, in an image including an animal, the animal may be shown present as part of an overall scene; alternatively, the image may be only of the animal. Similarly, in an image including non-animals, a single non-animalistic object may be shown; alternatively, a given scene not containing an animal may be displayed.

Following the display of each natural image, a blank screen is displayed on the computer screen for a period of 20±10 ms. The time interval for the display of each blank screen is varied within the range of 20±10 ms to prevent adaptation by the subject and may be referred to as the interstimulus interval. Each blank image acts as an interstimulus interval. Following the display of each blank image, a noisy mask comprising a noise image is displayed on the computer screen for a period of 250±100 ms. The stimulus onset asynchrony ranges from 60 to 180 ms accounting for the 100±50 ms time interval for the display of each natural image and for the 20±10 ms time interval for the display of the blank screen. Following the display of each masked image, a decision screen is displayed. Each decision screen is displayed for a predetermined duration during which time the subject may still input their response. Inputs may be a “YES” or “NO” response received from the subject during this time frame or alternatively, if an input is not received during the predetermined duration, an input of “NO RESPONSE” is recorded. Once the decision screen time interval has elapsed, the next natural image in the series is displayed to the subject and the process is repeated until the task is completed or terminated.

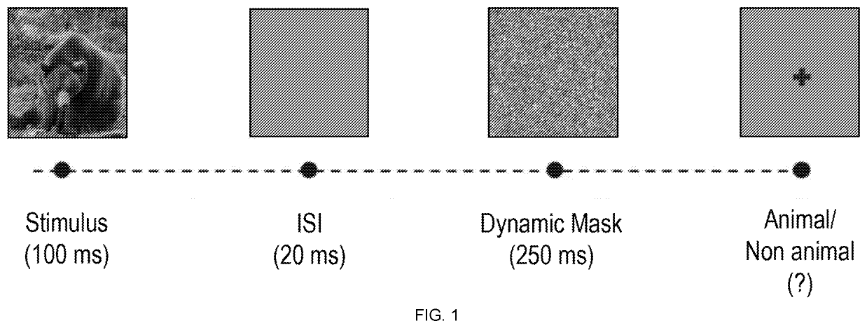

By way of example only, FIG. 1 illustrates an exemplary procedure for a computerised image categorisation task.

During the task as illustrated in FIG. 1, an individual greyscale natural image acting as a stimulus is displayed on a computer screen for 100 ms.

Following the display of the natural image, a blank screen is displayed on the computer screen for a period of 20 ms. Each blank screen acts as an interstimulus interval and is denoted as ISI in FIG. 1. Following the display of each blank screen a noisy mask comprising a noise image is displayed on the computer screen for a period of 250 ms. The stimulus onset asynchrony in this example is approximately 120 ms accounting for the 100 ms time interval for the display of the natural image and for the 20 ms display of the blank screen. Following the display of the masked image a decision screen is displayed. The decision screen is displayed for a predetermined duration during which time the subject may still input their response. Once the decision screen time interval has elapsed, the next natural image in the series is displayed to the subject and the process is repeated until the task is completed or terminated.

Following the display of each natural image, the objective of the subject is to assign the each given natural image to the correct category as quickly as possible. In other words, the goal of the task for the subject is to respond, as quickly and as accurately as possible, as to whether each displayed natural image satisfies a predetermined categorisation-based criterion. In an example, the subject is tasked with determining whether each displayed natural image contains an animal or not. For example, if the subject considers that the given natural image contains an animal, the subject is expected to select a designated response key representing “YES”; conversely, if the subject considers that natural image does not contain an animal, the subject is expected to select a designated response key representing “NO”. If an input is not received during the predetermined duration of display of the decision screen, an input of “NO RESPONSE” is recorded. A “NO RESPONSE” input is considered to be a miscategorisation by the subject. The resultant response data comprises a plurality of these inputs for each subject.

An input characteristic associated with the plurality of inputs is detected. The input characteristic comprises at least one of individual response times for each of the plurality of inputs, a cumulative response time for the plurality of inputs, a mean response time over all of the valid inputs, individual accuracy scores for each of the plurality of inputs, a cumulative accuracy score for the plurality of inputs or an overall accuracy score of the plurality of inputs.

Individual response times are defined by the duration of time from when a given natural image is first displayed to when the subject inputs the response. Individual response time includes the time required for visual processing and to input the response, thereby engaging a large volume of the cortex. These areas of the cortex may be affected in the early stages of brain disorders including Alzheimer's disease. Cumulative response times are defined by the sum of all of the individual response times received. A mean response time is defined by the sum of all the valid individual response times over the number of valid inputs.

Individual accuracy scores are defined by whether each individual input by the subject satisfies the predetermined categorisation criterion. In other words, each individual input is scored as either correct or incorrect. A cumulative accuracy score is defined by the number of correct and incorrect inputs. An overall accuracy is defined by the sum of the number of correct responses over the number of responses received.

In more detail, individual response times are detected and recorded for each valid input. The cumulative and mean response times are derivable from the individual response times. Preferably, the input is detected the a given natural image is replaced with a blank screen. In this manner, the task does not necessarily assess the recall ability of the subject; instead, the task intends to evaluate the ability of the subject to mentally process the natural images. The individual accuracy score indicates the ability of the subject to process each of the natural images, whilst the cumulative accuracy score or the overall accuracy score is indicative of the performance of the subject throughout the duration of the task.

The categorisation task undertaken by healthy and non-healthy training subjects and candidate subjects (e.g. candidates undertaking the task with no a priori knowledge of brain health status or brain biomarker status) are conducted in a similar manner and as described previously. For each group of subjects, completion of the task yields at least one set of response data comprising a plurality of inputs and an input characteristic associated with the plurality of inputs. In an exemplary categorisation task, approximately 100 natural images are displayed to the subject. In an alternative example, fewer natural images may be displayed; in a further example, additional images exceeding 100 may be displayed.

The display of a given natural image and the following blank image, masked image and decision screen may be considered to be a discrete trial within the categorisation task. Further, the display of a given natural image, followed by the blank image, masked image and decision screen is considered to be a second discrete trial within the categorisation task. As such, in an example where 100 natural images are displayed to the subject, the categorisation task comprises 100 individual trials. In a given categorisation task, the trials wherein the image includes an animal and wherein the image includes a non-animal are randomly displayed. Consequently, there is no given pattern at which the animal and non-animal images are displayed such that the subject is unable to predict the response to the next trial.

Preferably, each training subject and each candidate subject is provided with a preparation task implemented according to the above process before the start of the main categorisation task. This allows the subject to become familiar with the task prior to the start time, thereby potentially removing any confusion for the subject at the onset of the task.

As described previously, in addition to the data from the categorisation task, a second set of data relating to data associated with each subject is received. The second set of data includes characteristic data comprising at least one parameter of a characteristic associated with the subject may be collected. Each set of characteristic data comprises at least one parameter that is indicative of a characteristic associated with the subject. In some examples, the subject is a healthy subject. In other examples, such as those described below, the subject is an unhealthy subject (i.e. having a known disease state).

The characteristic data comprises at least one of personal data, medical data, medical history data, family medical history data, lifestyle data, clinical data, or genetic data. In more detail, personal data may comprise any data relating to personally identifiable information including but not limited to, age, gender, biometric data, socioeconomic status, ethnicity and education. Medical data may comprise any data indicative of the health status of the subject. Medical history data may comprise any data indicative of the health status of the subject previously. Family medical history data may comprise any data indicative of the healthy status of the blood relatives of the subject previously. Lifestyle data may comprise any data relating to the general wellbeing and style of living of the subject, including but not limited to, smoker status, general sleep habits, fitness levels, diet, and alcohol consumption. Clinical data comprises any data collected during clinical care or research. Genetic data may comprise any data relating to the genetic characteristics of the subject. The characteristic data can be scored to produce a parameter that is descriptive of that given characteristic.

The characteristic data may be derived from the results of a standardised cognitive assessments completed by the subject. Examples of such cognitive assessments include, but are not limited to, the Montreal Cognitive Assessment, the Mini-Mental State Exam, the Mini-Cog, the Addenbrooke's Cognitive Examination or any other standardised test designed to measure mental function.

In additional or alternative examples, the characteristic data may be derived from questionnaires completed by the subject. The questionnaire may include questions relating to personal information, medical data, medical history, family medical history, lifestyle of the subject or clinical data relating to already diagnosed conditions or results from medical tests. Additionally or alternatively, characteristic data may be extracted from wearable technology or tracking devices specifically designed for that purpose e.g. pedometers or fitness and exercise apps.

Additionally or alternatively, the characteristic data may comprise a genetic status of the subject. As a consequence, characteristic data may be extracted from genetic tests. By way of example, the subject may have undergone genetic testing to determine the status of the APOE gene. It is thought that the APOE gene has three variants, namely variants 2, 3 and 4. A copy of each gene is inherited from each parent. Subjects with one or two copies of variant 4 of the APOE gene have been shown to have an increased likelihood of developing Alzheimer's disease. In this example, the status of the APOE gene having two copies of variant 4 is received and processed. This allows a parameter indicative of the APOE status of the subject to be computed. This may be in terms of the relative risk of developing Alzheimer's disease based on the detected variants. It is apparent to one of ordinary skill in the art that the status of other genes may be tested for and included in the analysis.

The at least one set of characteristic data may be collected prior to the start of the categorisation task; for example, the characteristic data may be collected at a time interval of one week before the subject undertakes the task. Additionally or alternatively, characteristic data may be collected at the first time point at which the categorisation task is completed. Additionally or alternatively, the at least one set of characteristic data relating to the subject may be collected following completion of the categorisation task; for example, the characteristic data may be collected at a time interval of one week after the task has been completed.

Optionally, the subject may additionally complete the categorisation task at Nadditional time points. Additional sets of characteristic data may be collected before or after the completion of the additional categorisation tasks, or at the same time point at which these tasks are conducted. The one of ordinary skill in the art appreciates that the number of task completions and number of sets of characteristic data may match, but they need not. By way of example, a subject may complete the task three times with the characteristic data being collected at the first time point. In another example, a subject may complete the task three times with the characteristic data being collected at three different time points. In another example, a subject may complete the task three times with the characteristic data being collected at five different time points.

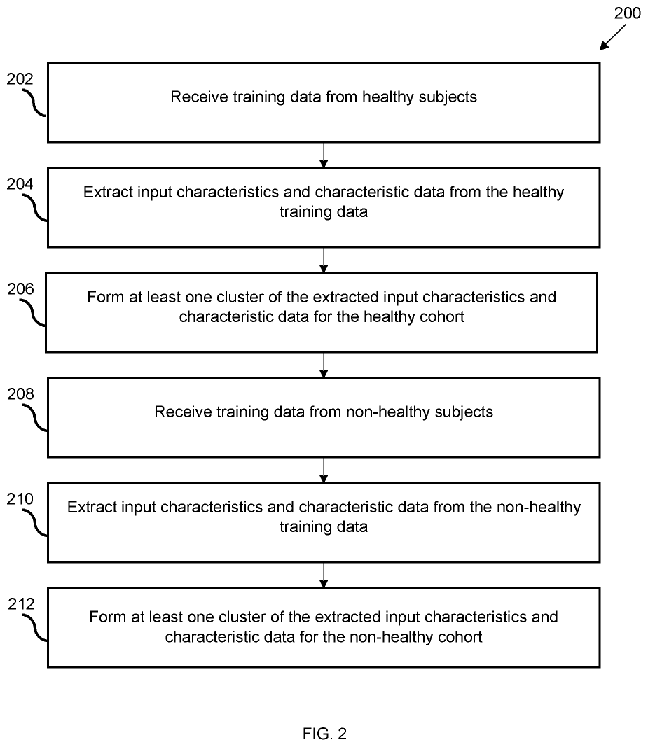

FIG. 2 is a flow-chart depicting an exemplary method 200 for collecting and analysing training data from a first cohort of healthy subjects and from a second cohort of non-healthy subjects.

At block 202, training data from a cohort of healthy subjects is received. The cohort of healthy subjects comprises a plurality of healthy subjects, each without a formal diagnosis of the presence of a brain disorder. The training data comprises a plurality of sets of characteristic data and a plurality of sets of response data, each labelled with the known brain biomarker status of the training subject. Each set of characteristic data and response data may be collected and collated as described previously.

For the healthy cohort, each set of response data comprises a plurality of inputs input by a healthy subject and at least one input characteristic associated with these inputs. Each input comprises a determination as to whether or not each image of a series of natural test images displayed satisfies at least one predetermined categorisation criterion. The input characteristic comprises individual response times for each of the plurality of inputs, a cumulative response time for the plurality of inputs, a mean response time, individual accuracy scores for each of the plurality of inputs, a cumulative accuracy score for the plurality of inputs or an overall accuracy score of the plurality of inputs. Each set of characteristic data comprises at least one parameter that is indicative of a characteristic associated with the healthy subject. The characteristic data comprises at least one of personal data, medical data, medical history data, family medical history data, lifestyle data, clinical data, or genetic data; these have been described in more detail previously.

At block 204, the input characteristics and the characteristic data are extracted from the healthy cohort training data.

At block 206, the extracted input characteristics and the extracted characteristic data are used to form at least one cluster for the healthy cohort. The vectors of each of the extracted components (i.e. the extracted input characteristics and the extracted characteristic data) are used for the clustering, wherein the centre of each cluster is defined by a centroid. In an example, the extracted input characteristics and the extracted characteristic data are used to form separate clusters. In an alternative example, the extracted input characteristics and the extracted characteristic data are used to form a single cluster by combining the vectors of the extracted data.

Optionally, model reference data and model characteristic data may be respectively derived from the reference response data and the reference characteristic data. A linear regression model can be fitted between the extracted input characteristics and the image statistics of the images in the training task in order to predict the input characteristic data for a training subject for different images. The predicted input characteristics may be referred to as model reference data. Similarly, a linear regression model can be fitted between the extracted characteristic data and the image statistics of the images in the training task in order to predict the characteristic data for a training subject for different images. The predicted characteristic data may be referred to as model characteristic data. Alternatively, the extracted input characteristics and the characteristic data can be combined; a linear regression model can be fitted between this combined data and the image statistics of the images in the training task in order to predict the input characteristic and characteristic data for a training subject. The modelling process is described in greater detail below.

The one of ordinary skill in the art appreciates that the clustering methods as described herein are also applicable to the predicted values of the input characteristics and the predicted values of the characteristic data. The sets of reference response data may comprise the actual response data or the actual response data in combination with the predicted response data. The sets of characteristic data may comprise the actual characteristic data or the actual characteristic data in combination with the predicted response data. The one of ordinary skill in the art appreciates that using these various types of data sets various types of clusters may be generated.

At block 208, training data for a cohort of non-healthy subjects is received. Each of the non-healthy subjects is a subject who has received a formal diagnosis indicating the presence of at least one brain disorder. Additionally or alternatively, each non-healthy subject may be a subject who has reported signs of early-onset symptoms such a memory loss, confusion or difficulties with concentration. For the non-healthy cohort, each set of response data and each set of characteristic data may be as described above with respect to the healthy cohort but is instead acquired from a plurality of non-healthy subjects.

At block 210, the input characteristics and the characteristic data are extracted from the non-healthy cohort training data.

At block 212, the extracted input characteristics and the extracted characteristic data are used to form at least one cluster for the non-healthy cohort. The vectors of each of the extracted components are used for the clustering. In an example, the extracted input characteristics and the extracted characteristic data are used to form separate clusters. In an alternative example, the extracted input characteristics and the extracted characteristic data are used to form a single cluster by combining the vectors of the extracted data. In each of these examples, the centre of each cluster is defined by a centroid.

By way of example only, the method is described in more detail in relation to the non-healthy cohort. Training data from a cohort of non-healthy subjects is received. The training data comprises a plurality of sets of response data and a plurality of sets of characteristic data relating to non-healthy subjects. Each of these sets of response data and characteristic data are labelled with a known brain biomarker status of the non-healthy training subject. The response data comprises an input and an input characteristic. In this example, the input characteristic comprises the overall accuracy score at which the non-healthy subjects performed the categorisation task and the characteristic data comprises the APOE status of each subject. Each subject has two copies of the APOE gene, with three known types such that there are six known possible combinations. E2/E2 is associated with the lowest risk of Alzheimer's disease, whilst E4/E4 is associated with the greatest risk of developing Alzheimer's disease. The relative risk of the remaining combinations is known; using this knowledge, a parameter representative of the characteristic can be computed. In this example, the overall accuracy score vectors and the APOE status vectors are combined in order to form a single cluster for the non-healthy cohort.

The one of ordinary skill in the art appreciates that the method described above and further detailed in the example is also applicable to the healthy cohort and, further, to both the healthy cohort and non-healthy cohort where the examples of the input characteristic and the characteristic data vary. It is also appreciated that additional input characteristics and characteristic data may be collected, extracted and clustered for each cohort. The vectors of the additional extracted data may be combined to form a single cluster or alternatively, separate clusters for each set of additional extracted data may be formed.

It is also appreciated that any number and combination of components and extracted features may be used and clustered. New training data may be continuously added to the established clusters to progressively develop the library of data against which candidate subjects may be assessed. Additionally, once the brain biomarker status of the candidate subject has been obtained, the candidate response data and candidate characteristic data may be added to the training data set such that the reference response data includes the candidate response data and the reference characteristic data includes the candidate characteristic data.

Further, in some examples, the training subjects may be categorised into sub-groups based on the characteristic data and/or the input characteristics or the predicted values thereof. By way of example, the detected input characteristic may be an accuracy score. As described previously, the accuracy score may comprise each individual accuracy score for each input.

Alternatively, the accuracy score may comprise a cumulative accuracy score summed over all of the received inputs. Alternatively, the accuracy score may comprise a mean accuracy score.

In this example, the accuracy score may be used to categorize participants into sub-groups on the basis of their accuracy score. Consider a cohort of participants with accuracy scores ranging from 52% to 94%. Sub-groups of accuracy scores falling between 50 to 60%, 60 to 70%, 70 to 80%, 80 to 90% and 90 to 100% may be formed and the subjects assigned to each of these sub-groups based on their relative scores. The related response data and characteristic data, actual and/or predicted, may then be clustered according to the methods described previously within each of these respective sub-groups. The one of ordinary skill in the art appreciates that the sub-groups may vary, and that the relative percentage ranges of such sub-groups may also be subject to variation.

In an example, the training data is processed. In an example, the processing of the training data may comprise extracting the data points from the training data. The ‘explainability’ or the degree of how informative a given data point is can be predetermined. In additional or alternative examples, the processing of the training data may comprise organising the training data into a suitable format for the machine learning model. The person skilled in the art appreciates that there are numerous means by which the data may be organised.

Optionally, each set of training data may be assessed for quality. In an example where the input characteristic comprises the overall accuracy scores, the scores may be used as a control measure to assess the relative attentiveness of the subject. By way of example, if a given overall accuracy score is lower than a predetermined threshold, it may be indicative of the subject not engaging in the task and that the subject seems to be responding in a random manner. If this is detected, the response data of the subject is deemed to be invalid and is not included in the training set.

Patterns of measured input characteristics and measured parameters of the characteristic data are defined in the training phase in the clusters for both healthy and non-healthy cohorts. For each training subject, each input characteristic and each characteristic data parameter is represented by a vector to a set of the displayed images, the vector having the same length as the number of displayed images. The input characteristic vectors and the characteristic data vectors are used to cluster the extracted data. The extracted data can be used as reference response data and reference input characteristic data against which corresponding candidate response data and corresponding candidate input characteristic data can be compared.

Optionally, in an additional example, model reference response data and model reference input characteristic data can be respectively derived from the reference response data and the reference input characteristic data. In the training phase, for each displayed image, the statistical properties are calculated and concatenated in a vector. The image statistics may comprise gamma and beta of Weibull distribution fitted to the edge of the histogram of the images, entropy, and Fourier slope and intercept of the images. Each displayed image has a vector of length five, referring to each of the five image statistics. For each image in the series, a Fourier analysis and entropy calculation are performed. A Weibull distribution is fitted. For the Fourier analysis, the Fourier slope and Fourier intercept are obtained. The image entropy is obtained from the entropy calculating. Parameters gamma and beta are obtained from the Weibull distribution.

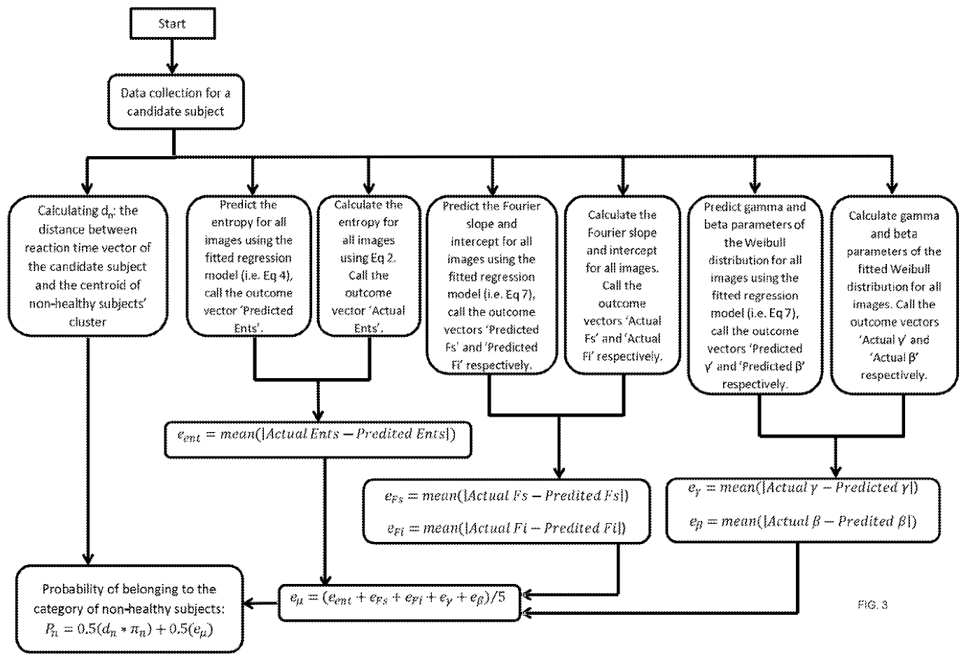

The five statistical properties of the images are discussed in more detail with reference to FIG. 3. FIG. 3 is a flow diagram illustrating the above five statistical properties. The first statistical property is entropy. The entropy of an image is a measure of the amount of information it contains. The formulation for calculating the entropy is:

−Σ(pᵢ*log (pᵢ)) (5)

where:
i is a state; and
pᵢ is the probability of that state.

To estimate the entropy of an input image I, the method as proposed by Chandler and Field is used (Chandler, D. M. and Field, D. J. (2007) ‘Estimates of the information content and dimensionality of natural scenes from proximity distributions’, Journal of the Optical Society of America A, 24(4), pp. 922-941).

A group, N, of 16 images are selected from the same category that image I belongs to. For image I and for each image in group N, 2¹⁴ non-overlapping patches of 8×8 pixels are extracted from top-left to the bottom-right of each image.

Entropy is calculated using the following equation:

$$h ( x ) ≅ C {\sum }_{m = 1}^{M} {log}_{2} {D}_{N , m} + {log}_{2} \frac{{A}_{q} N}{q} + \frac{ρ}{ln 2} \qquad ( 6 )$$

where:
C is a constant=

$$f ( x ) = c \cdot {exp ( \frac{x - μ}{β} )}^{γ} \qquad ( 7 )$$

in which q=64 because of 8×8 patches;
M=2¹⁴ total number of patches extracted from each image; and
D_{N,m} represents the minimum Euclidian distance between each patch of image I extracted from group N;

$${A}_{q} = \frac{q {π}^{\frac{q}{2}}}{gamma ( \frac{q}{2 + 1} )} ,$$

where gamma(x) is a gamma function defined by

$${\int }_{0}^{∞} {t}^{x} {e}^{- t} \frac{dt}{t} ;$$

and
ρ is the Euler constant, approximately equal to 0.577.

The second and third statistical properties are gamma and beta. These are derived from the Weibull distribution. The edge histogram of a natural scene follows a Weibull distribution of the form in equation (3), which is a continuous probability distribution with two free parameters called beta (β) and gamma (γ).

$$f ( x ) = c \cdot {exp ( \frac{x - μ}{β} )}^{γ} \qquad ( 7 )$$

where:
c is a normalization constant allowing f to be a probability distribution function,
μ is the origin of the contrast distribution,
β represents scale parameters of the Weibull distribution; and
γ represents shape parameters of the Weibull distribution.

The edge histogram for a given image is calculated by running an edge detector on the image and then counting the frequency of edges in different orientations. This gives the histogram edges in different orientations for the given image.

The fourth and fifth statistical properties are the Fourier slope and the Fourier intercept. Fourier statistics for an image can be derived by calculating the intercept and slope of a line fitted to the power spectrum of the image. The power spectrum for a given image can be derived by transferring the image to the Fourier space (e.g. using fast Fourier transform), and then plotting the proportion of the power of the signal falling within given frequency bins.

Mappings between the input characteristics and the statistical properties of each displayed image, and between the characteristic data and the statistical properties of each displayed image are learnt. Alternatively, a mapping between the image statistics and the combined input characteristics and the characteristic data may be learnt. These mappings are respectively learnt by fitting linear regression models between the input characteristic data and the image statistics, between the characteristic data and the image statistics, or between the image statistics and a combined data set comprising the characteristic data and the input characteristics.

By way of example only, for entropy and the input characteristic data, the linear regression model is the following:

Enᵢ=ICᵢ*Wᵢ+εᵢ (8)

where:
ICᵢ is the input characteristic data value; and
εᵢ is the error term.

Enᵢ=[en₁, en₂, . . . , enₗ, . . . , enₙ] (9)

where:
Enᵢ is the entropy of image i; and
n is number of subjects.

To fit the model, first all Enₗ are initialized to the calculated entropy for image i, using equation (6).

Fitting the regression model for the input characteristic data and the entropy means finding Wᵢ in equation (8) for each image.

Equation (8) can be generalised for an arbitrary image statistic for the input characteristic as the following:

Istᵢ=ICᵢ*Wᵢ+εᵢ (10)

where:
ICᵢ is the input characteristic data value; and
εᵢ is the error term.

Similarly, equation (8) can be generalised for an arbitrary image statistic for the characteristic data as the following:

Istᵢ=CDᵢ*Wᵢ+εᵢ (11)

where:
CDᵢ is the characteristic data parameter value; and
εᵢ is the error term.

Equation (8) can be generalised for an arbitrary image statistic for the combined input characteristic and characteristic data as the following:

Istᵢ=Dᵢ*Wᵢ+εᵢ (12)

where:
Dᵢ is the combined characteristic data value and the input characteristic value; and
εᵢ is the error term.

In each case, equation (9) can be generalised for an arbitrary image statistic as the following:

Istᵢ=[ist₁, ist₂, . . . , istₗ, . . . , istₙ] (13)

where:
Istᵢ is one of the mentioned image statistics calculated for image i; and
n is number of subjects.

As described above, the clustering of the input characteristic data and the characteristic data allows distinct patterns of input characteristics and the characteristic data for healthy and non-healthy subjects to be defined. These patterns may be used to predict input characteristic data and characteristic data for images not contained in the original training task categorisation for both healthy and non-healthy cohorts. A model is fitted to the measured input characteristics and the measured characteristic data based on the image statistics of the images that were contained in the training task. As described previously, the image statistics may comprise gamma and beta of Weibull distribution fitted to the edge of the histogram of the images, entropy, and Fourier slope and intercept of the images.

Using the relationship defined between the measured input characteristics and the image statistics in the displayed images, the values of the input characteristics for unseen natural images can be predicted for the training subjects, producing model reference response data. Similarly, using the relationship defined between the measured characteristic data parameters and the image statistics in the displayed images, the values of the characteristic data parameters for unseen natural images can be predicted for the training subjects, producing model reference characteristic data. As a consequence, the analysed training data set may comprise at least one of reference response data or model reference response data derivable from the reference response data and at least one of reference characteristic data or model reference characteristic data derivable from the reference characteristic data.

In more detail, to fit the model for both healthy cohort response data and non-healthy cohort response data, all image statistics are calculated for an unseen series of natural images. An optimal weight vector for each image statistic is computed using the least square error approach. The computation comprises mapping each of the image statistics relating to the images displayed in the training task to each of the measured input characteristics. Additionally or alternatively, the computation may comprises mapping each of the image statistics relating to the images displayed in the training task to each of the measured characteristic data parameters. The least square error approach minimises the sum of the squared residuals in order find the best curve that fits to a set of points, with the residual being the difference between an observed value and the fitted value provided by the model. Using the best estimated weight vector, the input characteristics and characteristic data for the healthy subjects and the non-healthy subjects can be predicted for an unseen series of images based on the calculated image statistics. As a consequence, modelled response data and modelled characteristic data for new and unseen images can be generated.

In some optional examples, additional parameters representative of the inputs may be derived. As described previously, these additional parameters comprise at least one of a speed score, a test index, an accuracy performance slope, a speed performance slope, a test index performance slope, an accuracy score per subcategory of images, a speed score per subcategory of images, a test index score per subcategory of images, an attention score, a consecutive accuracy score or a consecutive inaccuracy score. The one of ordinary skill in the art appreciates that the vectors of each of these additional parameters may be used to include these additional parameters within the at least one cluster formed for each of the healthy and non-healthy cohorts according to the methods as described herein. Further, it is appreciated that predicted values for each of these parameters may be estimated according to the modelling methods as described herein.

Mappings between the input characteristics and the known brain biomarker status of each training subject, and between the characteristic data and the known brain biomarker status of each training subject are learnt. Alternatively, a mapping between the known brain biomarker statistics and the combined input characteristics and the characteristic data may be learnt. The one of ordinary skill in the art appreciates that these methods as described herein are also applicable to the predicted values of the input characteristics and the predicted values of the characteristic data. The sets of reference response data may comprise the actual response data or the actual response data in combination with the predicted response data. The sets of characteristic data may comprise the actual characteristic data or the actual characteristic data in combination with the predicted response data. The one of ordinary skill in the art appreciates the various combinations of which clusters may be formed, and as such, of which mappings may be generated.

The one of ordinary skill in the art appreciates that any suitable machine learning model may be used to perform map the input characteristic data and the known brain biomarker status, between the characteristic data and the known brain biomarker status or between the known brain biomarker status and the combined input characteristics and characteristic data.

In an example, these mappings may be respectively learnt by fitting linear regression models between the input characteristic data and the known brain biomarker status, between the characteristic data and the known brain biomarker status or between the known brain biomarker status and the combined input characteristics and characteristic data. In this example, for a given brain biomarker status and the input characteristic data, the linear regression model may be the following:

BSᵢ=ICᵢ*Wᵢ+εᵢ (14)

where:
ICᵢ is the input characteristic data value; and
εᵢ is the error term.

Additionally, in this example, for a given brain biomarker status and the characteristic data, the linear regression model may be the following:

BSᵢ=CDᵢ*Wᵢ+εᵢ (15)

where:
CDᵢ is the characteristic data parameter value; and
εᵢ is the error term.

Additionally, in this example, for a given brain biomarker status and the combined characteristic data and input characteristic data, the linear regression model may be the following:

BSᵢ=Dᵢ*Wᵢ+εᵢ (16)

where:
Dᵢ is the combined characteristic data value and the input characteristic value; and
εᵢ is the error term.

In each of these cases demonstrated for this example, the following may apply:

BSᵢ=[bs₁, bs₂, . . . , bsₗ, . . . , bsₙ] (17)

where:
BSᵢ is one of the known brain biomarker statuses for the training subjects; and
n is number of subjects.

Fitting the regression models for the brain biomarker status and the input characteristics, the characteristic data or the combined data means finding Wᵢ in equations (14), (15) and (16) respectively.

The one of ordinary skill in the art appreciates that mappings between the predicted values of the input characteristics and the characteristic data and the known brain biomarker status of the given training subject can be performed in a similar manner as described herein.

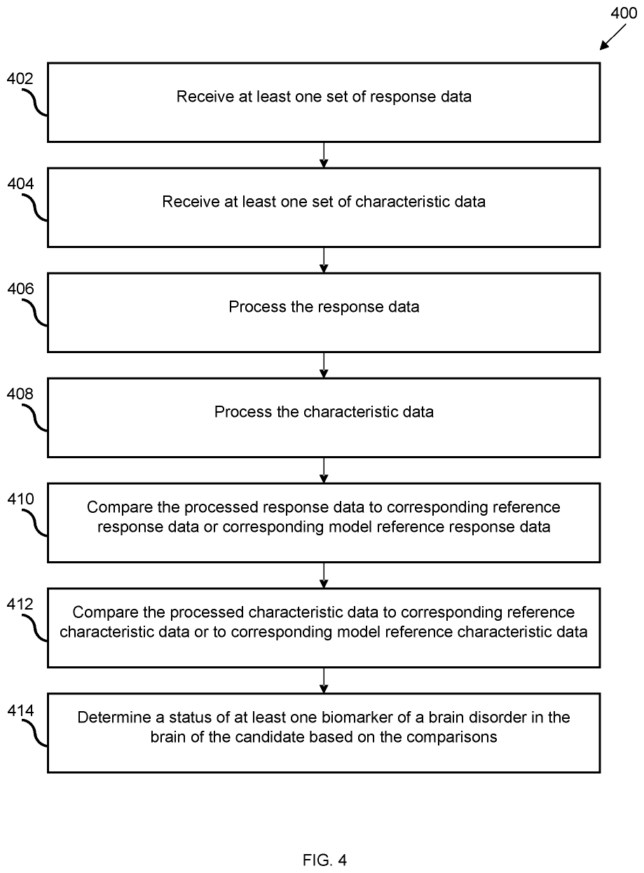

FIG. 4 illustrates an exemplary method 400 for predicting brain biomarker status. At block 402, at least one set of response data is received. The response data comprises a plurality of inputs input by a subject and at least one input characteristic associated with one or more of the plurality of inputs. Each of the inputs comprises a determination as to whether or not each image of a series of natural test images satisfies at least one predetermined categorisation criterion.

The input characteristic comprises data representative of at least one feature associated with one or more of the inputs. The input characteristic comprises at least one of individual response times for each of the plurality of inputs, a cumulative response time for the plurality of inputs, a mean response time over all of the valid inputs, individual accuracy scores for each of the plurality of inputs, a cumulative accuracy score for the plurality of inputs or an overall accuracy score of the plurality of inputs. Each of these measures is discussed in greater detail above with regard to the training subjects and is applicable to the candidate subject.

The characteristic data comprises at least one of personal data, medical data, medical history data, family medical history data, lifestyle data, clinical data, or genetic data. In more detail, personal data may comprise any data relating to personally identifiable information including but not limited to age, gender, biometric data, socioeconomic status, ethnicity and education. Medical data may comprise any data indicative of the health status of the subject. Each of these types of data are discussed in greater detail above with regard to the training subjects and is also applicable to the characteristic data received from the candidate subject.

Optionally, similarly to the training subjects, additional parameters further representative of the inputs may be derived from the input characteristics. The additional parameters may comprise a speed score, a test index, an accuracy performance slope, a speed performance slope, an accuracy score per subcategory of images, a speed score per subcategory of images, a test index score per subcategory of images, an attention score or a consecutive accuracy score. Each of these measures is discussed in greater detail with respect to the training subjects and is also applicable to the candidate subject.

At block 404, at least one set of characteristic data is received. The characteristic data comprises at least one parameter indicative of a characteristic associated with the subject.

At block 406, the at least one set of response data is processed to extract the at least one input characteristic, thereby generating at least one set of processed response data.

At block 408, the at least one set of characteristic data is processed.

At block 410, a first comparison is performed by comparing the at least one set of processed response data to corresponding reference response data or model reference response data. The model reference response data is derivable from reference response data. The reference response data comprises the actual measured input characteristic data collected for each subject in the healthy and non-healthy cohorts in the training phase. The model reference response data comprises the estimated input characteristic data derived from the model fitted to the actual measured input characteristic data.

Optionally, the comparison of the measured input characteristics of the candidate against the reference response data or the modelled reference response data allows a general assessment as to whether the candidate has, or is likely to develop, a brain disorder to be made.

At block 412, a second comparison is performed by comparing the at least one set of processed characteristic data to reference characteristic data or to model reference characteristic data. The model reference characteristic data is derivable from reference response data. The reference characteristic data comprises the actual measured characteristic data parameter or parameters collected for each subject in the healthy and non-healthy cohorts in the training phase. The model reference characteristic data comprises the estimated parameter of the characteristic data derived from the model fitted to the actual measured parameter of the characteristic data.

At block 414, a status of at least one biomarker of a brain disorder in the brain of the subject is determined based on the first comparison and the second comparison. The status of the at least one biomarker of the brain disorder may comprise at least one of biomarker positivity, concentration, volume or size of the at least one biomarker in the brain of the subject. The at least one brain biomarker may comprise any suitable neuroimaging marker, fluid biomarker, biochemical marker, or other structural or functional marker in the brain. In some examples, the at least one brain biomarker may comprise at least one of neurofilament light chain, amyloid beta or Tau.

It may be determined that the status of the at least one biomarker exceeds a predetermined threshold or is lower than a predetermined threshold.

An indication of a risk of the subject developing at least one brain disorder may be determined based on the determined status of the at least one biomarker. A determination as to the indication of the risk of a subject developing at least one brain disorder may further be based on the determined status of the at least one brain biomarker exceeding or being lower than a predetermined threshold.

An indication of the presence of at least one brain disorder based on the determined status of the at least one biomarker may be determined. A determination as to the indication of the presence of at least one brain disorder may be further based on the determined status of the at least one brain biomarker exceeding or being lower than a predetermined threshold.

An indication as to the severity of the at least one brain disorder that is determined to be present may be determined based on at least the determined status of the at least one brain biomarker. A determination as to an indication of the severity of the at least one brain disorder that is determined to be present may be further based on the determined status of the at least one brain biomarker exceeding or being lower than a predetermined threshold.

An indication as to the prognosis of the at least one brain disorder that is determined to be present may be determined based on at least the determined status of the at least one brain biomarker. A determination as to the indication of the prognosis of the at least one brain disorder that is determined to be present may be further based on the determined status of the at least one brain biomarker exceeding or being lower than a predetermined threshold.

In any of these examples, the at least one brain disorder may be at least one of Alzheimer's Disease, cerebrovascular dementia, mild cognitive impairment, frontotemporal dementia, dementia with Lewy Bodies, multiple sclerosis, motor neurone disease, Parkinson's disease, attention deficit hyperactivity disorder, primary mental illness Huntington's disease, depression, post-traumatic stress disorder or brain injury.

Optionally, at least one therapeutic to be administered may be determined based on the determined status of the at least one brain biomarker. Additionally or alternatively, at least one non-pharmacological treatment to be implemented may be determined based on the determined status of the at least one brain biomarker.

As described previously, the training subjects may be categorised into sub-groups based on the characteristic data and/or the input characteristics and/or the predicted values thereof. Clusters may be formed within these sub-groups according to the techniques described previously. The candidate data may then be compared to these sub-group clusters.

As described previously, and by way of example, the accuracy score may be used to categorise training subjects into sub-groups on the basis of their accuracy score. By way of example, consider a cohort of training subjects achieving accuracy scores ranging from 52% to 94%. Sub-groups of accuracy scores falling between 50 to 60%, 60 to 70%, 70 to 80%, 80 to 90% and 90 to 100% may be formed and the training subjects assigned to each of these sub-groups based on their relative scores. The related response data and characteristic data, actual and/or predicted, may be clustered within these sub-groups. If a candidate subject is determined to have achieved an accuracy score of 72%, the resultant comparisons may be conducted against the reference response data, reference characteristic data, modelled reference response data or modelled reference characteristic within the 70 to 80% sub-group clusters.

Optionally, the candidate subject completes the categorisation task at any number, Nadditional, of additional time points. It is appreciated that the candidate subject may complete the task at one additional time point such that they have completed two tasks in total. It is appreciated that the candidate may complete further additional tasks accordingly.

In any of these examples, the time interval between each completion of the categorisation task may be separated by a predetermined time frame. The time frame of the separation may range from hours to weeks to years. In examples where the task is undertaken more than two times, it is appreciated that the time interval between the tasks may be the same or it may be different. By way of example, the second task may be undertaken one week after the first, whilst the third task may be undertaken a matter of hours or days after the second task, one week after the second task or more than one week after the second task.

Optionally, where a candidate undertakes a task at a further time point or time points, the method as depicted in FIG. 4 may be repeated for each of the completed tasks. As such, the response data associated with the N_{additional} tasks is received and processed. The characteristic data associated with the subject undertaking the N_{additional} tasks is received and processed. The processed N_{additional} response data is compared to the reference response data or to model reference response data. The processed N_{additional} characteristic data is compared to the corresponding reference characteristic data or to model reference characteristic data.

Optionally, where a candidate undertakes a plurality of tasks, the response data associated with each given task is compared to the response data associated with the other remaining tasks. This comparison enables a change in the response data over time to be determined. A trend or pattern in the response data over time can be deduced. In an example, a candidate undertakes a first categorisation task at a first timepoint, t₁. A candidate undertakes further tasks at further time points denoted by tₙ, where n denotes the number of tasks completed. Where a candidate has completed four tasks (i.e. n=4), response data for each of these tasks is received and processed. Characteristic data associated with the candidate is received. The characteristic data need not be acquired at each time point. Alternatively, a new set of characteristic data may be acquired and received at each time point. In further examples, additional sets of characteristic data need not be acquired at the same time point as the completion of the categorisation tasks.

Any characteristic data that is available at t₄ is processed. In an example, the processing of the characteristic data may comprise extracting the most informative and/or explainable data points from the characteristic data to be passed to subsequent stages of the computer-implemented method. The explainability or the degree of how informative a data point is can be predetermined during the training phase. In additional or alternative examples, the processing of the characteristic data may comprise organising the data into a suitable format for the machine learning model. The person skilled in the art appreciates that there are numerous means by which the data may be organised.

A first comparison is performed in which the response data for each of the four time points is compared to the reference data or the model reference data. The characteristic data that is available is compared to the corresponding reference characteristic data or the corresponding model reference data. The change in the response data over time is computed based on a comparison between each of the four sets of response data. A trend or pattern in the response data over time can be inferred based on the computed change in the response data.

As described previously, the sets of reference response data, modelled response data, reference characteristic data and modelled characteristic data may have been mapped to the status of a brain biomarker; as such, based on the comparison of the candidate response data and candidate characteristic data to the corresponding set of reference data or modelled reference data, a status of a brain biomarker is determinable.

As described previously, a machine learning model may be used to map the input characteristics, the characteristic data and/or the combined input characteristics and characteristic data to the brain biomarker status. These mappings allow a brain biomarker status for a candidate subject to be predicted.

In an example, the mapping is established by fitting a linear regression model. The resulting mapping may be used to estimate the brain biomarker status of the candidate subject. By way of example, a linear regression model fitted according to equations (16) and (17) based on the combined characteristic data and the input characteristics may be used to estimate the brain biomarker status of the candidate. In an additional or alternative example, a linear regression model may be fitted according to equations (14) and (17), and the resulting mapping may be used to estimate the brain biomarker status of the candidate. In a further additional or alternative example, a linear regression model may be fitted according to equations (15) and (17), and the resulting mapping may be used to estimate the brain biomarker status of the candidate.

For a given candidate subject, the respective correlation distances between the individual vectors of the input characteristics and the characteristic data and the centroids of the respective clusters are generated for the healthy and non-healthy cohorts. Additionally or alternatively, the correlation distance between the combined vectors of the input characteristics and the characteristic data and the centroids of the single cluster for each of the healthy and non-healthy cohort are generated. As mappings between the brain biomarker status and the input characteristics, the characteristic data, the predicted input characteristics, the predicted characteristic data and/or any suitable combination thereof may have previously been defined, based on the relative correlation distance or distances to the centroids of clusters, a brain biomarker status for the candidate subject is able to be predicted.

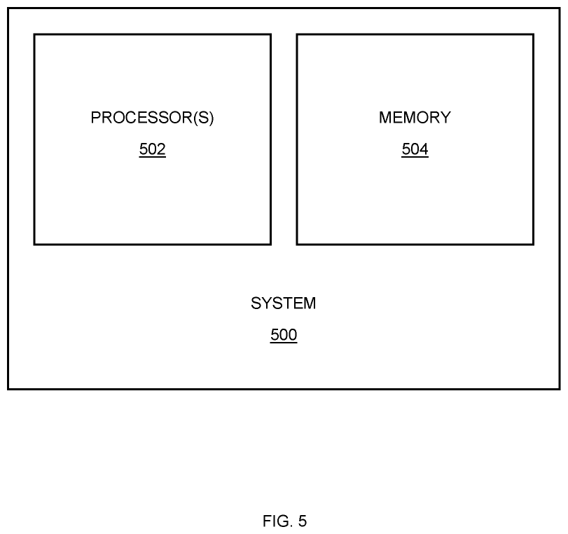

FIG. 5 depicts an exemplary system 500 implementable according to aspects of the present technology. System 500 comprises one or more processors 502 and a memory 504. The one or more processors 502 may be configured to execute instructions read from the memory 504.

The memory 504 may include computer-readable media. Computer readable media may be any available media that is accessible on a computer system and may comprise computer storage media and communication media. Computer storage media may comprise RAM, ROM, EEPROM, random-access memory (RAM), read-only memory (ROM), electrically erasable programmable read-only memory (EEPROM), flash memory, phase change memory (PRAM), statis random-access memory (SRAM), dynamic random-access memory (DRAM), flash memory, compact disk read-only memory (CD-ROM), digital versatile disks (DVD) or other optical storage, magnetic disk storage, magnetic tape or any other suitable medium that is useable to store data for access by a computer system. Communication media may comprise computer-readable instructions, data structures, program modules or data modulated in a data signal.

Any of the acts of any of the methods described herein may be implemented by the one or more processors configured with executable instructions that may be stored in the memory or on one or more computer-readable media.

Although FIG. 5 shows an exemplary system 500, it will be understood that other computing systems with varying aspects and components may be used.

The non-transitory computer-readable medium may have stored thereon a set of instruction which, when executed, cause the at least one processor to perform any of the acts of any of the methods as described herein.

The present technology may be implemented using computer programming techniques and through the use of software, hardware or firmware, or through any suitable combination of these. A program having computer-readable instructions may be provided within computer-readable media, resulting in a computer program product, implementable according to aspects of the present technology.

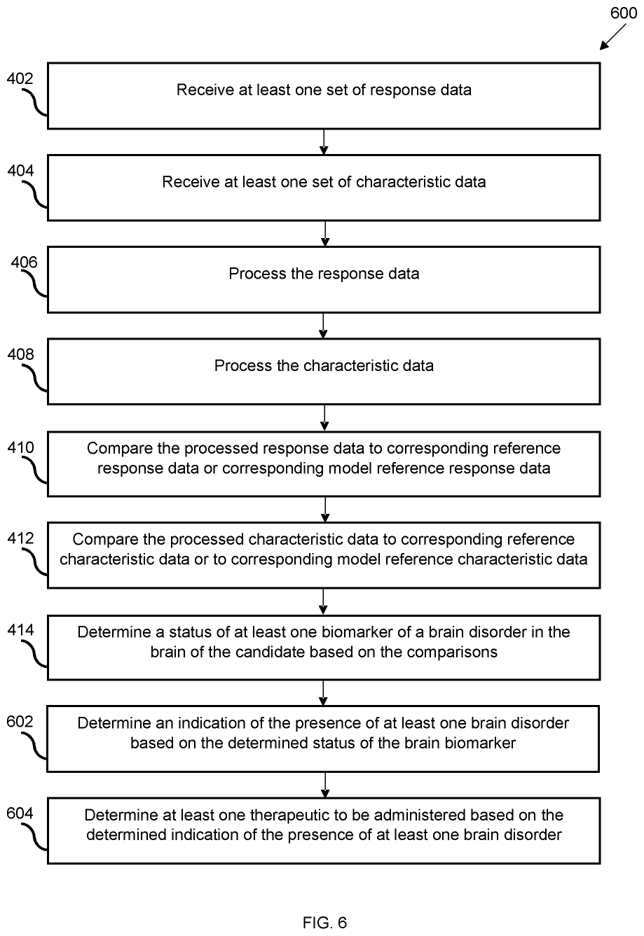

FIG. 6 illustrates an exemplary method of treatment according to aspects of the present invention.

Steps 402 to 414 are implemented as described previously with reference to FIG. 4.

At block, 602, based on the detected status of the brain biomarker, an indication of the presence of at least one brain disorder is determined. The biomarker may comprise at least one of neurofilament light chain, amyloid beta or Tau.

At block 604, based on the determined indication of the presence of at least one brain disorder, at least one therapeutic to be administered is determined.

Optionally, at block 606, the therapeutic is administered.

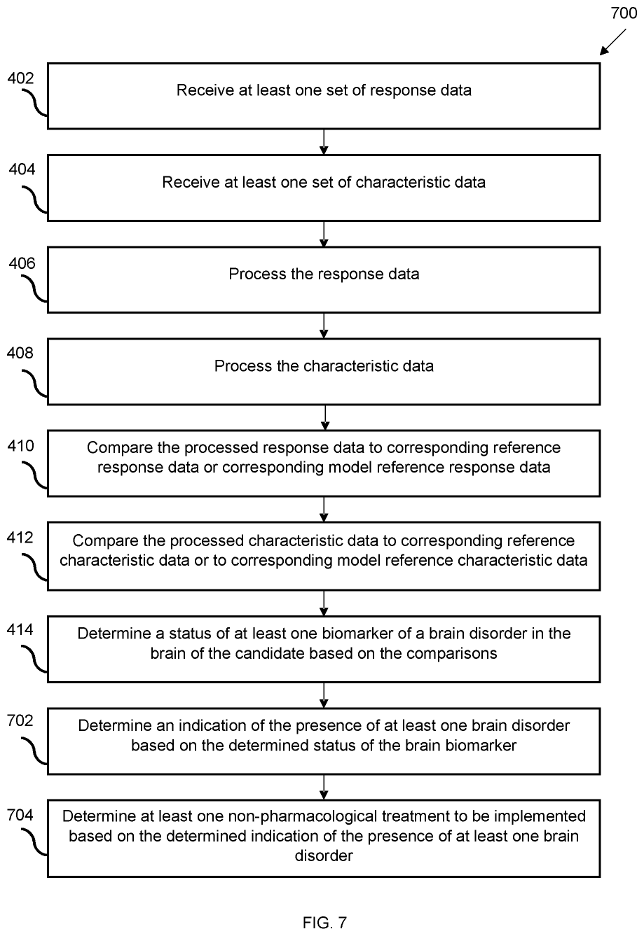

FIG. 7 illustrates an exemplary method 700 of treatment according to aspects of the present invention.

Blocks 402 to 414 are implemented as described previously with reference to FIG. 4.

At block, 702, based on the detected status of the brain biomarker, an indication of the presence of at least one brain disorder is determined. The biomarker may comprise at least one of neurofilament light chain, amyloid beta or Tau.

At block 704, based on the determined indication of the presence of at least one brain disorder, at least one non-pharmacological treatment to be implemented is determined.

Optionally, at block 706, the non-pharmacological treatment is implemented.

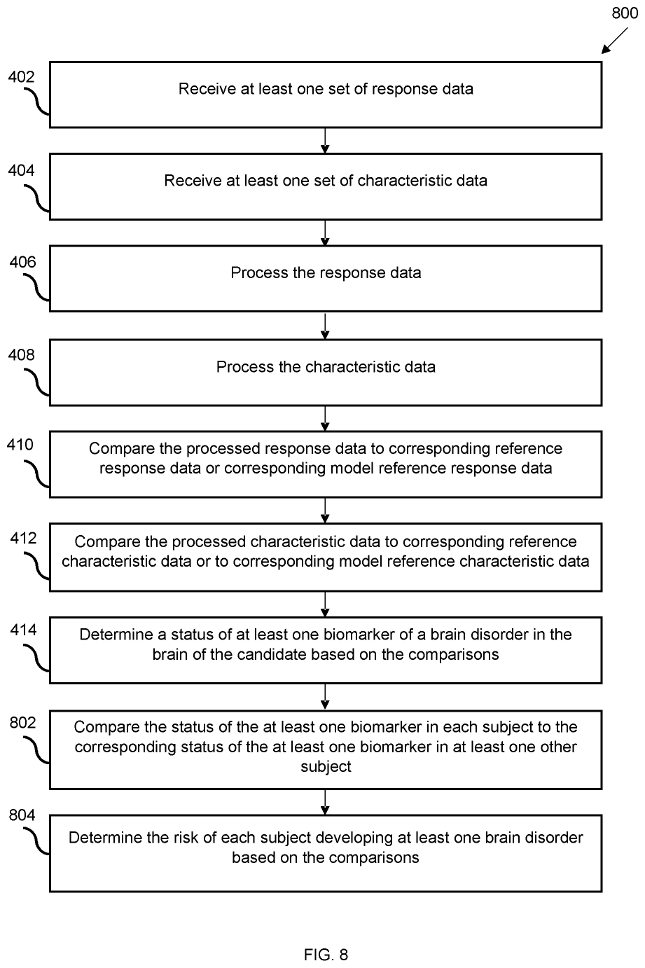

FIG. 8 illustrates an exemplary method 800 of screening potential patients.

Blocks 402 to 414 are implemented as described previously with reference to FIG. 4 for a plurality of subjects.

At block 802, a comparison of the status of the at least one biomarker of the brain disorder in each to the corresponding status of the at least one biomarker of the brain disorder in at least one other subject is performed.

At block 804, the risk of each subject developing at least one brain disorder is determined based on each of the comparisons.

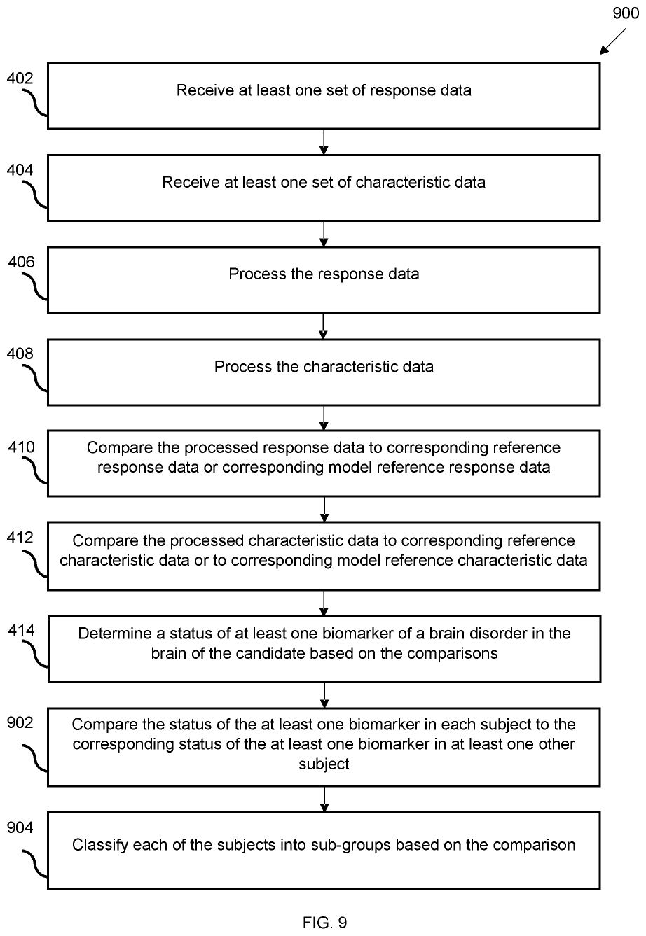

FIG. 9 illustrates an exemplary method 900 of patient stratification.

Blocks 402 to 414 are implemented as described previously with reference to FIG. 4 for a plurality of subjects.

At block 902, a comparison of the status of the at least one biomarker of the brain disorder to the corresponding status of the at least one biomarker of the brain disorder in at least one other subject is performed for each subject.

At block 904, each of the plurality of subjects is classified into a plurality of sub-groups based on the comparison.

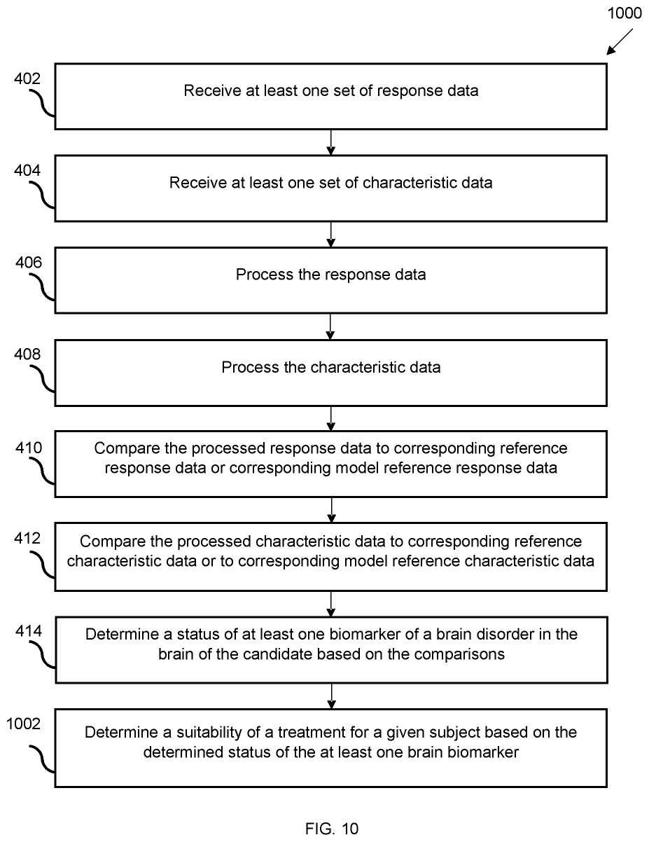

FIG. 10 illustrates an exemplary method 1000 for determining a suitability of a treatment for a given subject.

Blocks 402 to 414 are implemented as described previously with reference to FIG. 4 for the given subject.

At block 1002, based on the determined status of the least one brain biomarker, the suitability of the treatment for the given subject is determined.

### CLAUSES

Alternatively or in addition to the other examples described herein, further examples include:

Clause A. A computer-implemented method comprising: receiving at least one set of response data comprising a plurality of inputs by a subject and at least one input characteristic associated with the plurality of inputs, wherein each of the inputs comprises a determination as to whether or not each image of a series of natural test images displayed satisfies at least one predetermined categorisation criterion; receiving at least one set of characteristic data comprising at least one parameter indicative of a characteristic associated with the subject; processing the at least one set of response data; processing the at least one set of characteristic data; performing a first comparison by comparing the at least one set of processed response data to reference response data or to model reference data derived from reference response data; performing a second comparison by comparing the at least one set of processed characteristic data to corresponding reference characteristic data or to model characteristic data derived from corresponding reference characteristic data; and determining a status of at least one biomarker of a brain disorder in the brain of the subject based on the first comparison and the second comparison.

Clause B. The computer-implemented method of clause A wherein the at least one set of response data comprises a plurality of sets of response data each obtained at a different respective time point.

Clause C. The computer-implemented method of clause B wherein each different respective time point is separated by a predetermined time interval.

Clause D. The computer-implemented method of clauses B or C further comprising: comprises performing a third comparison by comparing each set of response data to each of the other respective sets of response data; and determining a change in response data over time based on the third comparison, wherein determining the status of the at least one biomarker of the brain disorder is further based on the determined change in response data.

Clause E. The computer-implemented method of any of clauses A to D, wherein the status of the at least one biomarker of the brain disorder comprises at least one of biomarker positivity, concentration, volume or size of the at least one biomarker in the brain of the subject.

Clause F. The computer-implemented method of any of clauses A to E, further comprising determining that the status of the at least one biomarker of the brain disorder exceeds a predetermined threshold.

Clause G. The computer-implemented method of any of clauses A to E, further comprising determining that the status of the at least one biomarker of the brain disorder is lower than a predetermined threshold.

Clause H. The computer-implemented method of any of clauses A to G, further comprising determining an indication of a risk of the subject developing at least one brain disorder based on the determined status of the at least one biomarker.

Clause I. The computer-implemented method of any of clauses A to H, further comprising detecting an indication of the presence of at least one brain disorder based on the determined status of the at least one biomarker.

Clause J. The computer-implemented method of claim I, further comprising determining at least one therapeutic to be administered based on the detected indication of the at least one brain disorder.

Clause K. The computer-implemented method of any of clauses A to J, further comprising determining at least one non-pharmacological treatment to be implemented based on the detected indication of the at least one brain disorder.

Clause L. The computer-implemented method of any of clauses H to K, wherein the brain disorder is at least one of Alzheimer's Disease, cerebrovascular dementia, mild cognitive impairment, frontotemporal dementia, dementia with Lewy Bodies, multiple sclerosis, motor neurone disease, Parkinson's disease, attention deficit hyperactivity disorder, primary mental illness or Huntington's disease.

Clause M. The computer-implemented method of any of clauses A to L, wherein the at least one biomarker comprises at least one of neurofilament light chain, amyloid beta or Tau.

Clause N. The computer-implemented method of any of clauses A to M, further comprising adding the at least one set of response data to the reference response data and the at least one set of characteristic data to the reference characteristic data once the biomarker status of the subject has been obtained.

Clause O. The computer-implemented method of any of clauses A to N, wherein the reference characteristic data comprises a first set of characteristic data pre-obtained from a plurality of subjects each with at least one brain disorder and a second set of characteristic data pre-obtained from a plurality of control subjects, and the reference response data comprises a first set of response data pre-obtained from a plurality of subjects each with at least one brain disorder and a second set of response data pre-obtained from a plurality of control subjects.

Clause P. The computer-implemented method of any of clauses A to O, wherein the characteristic data comprises at least one of personal data, medical data, medical history data, family medical history data, lifestyle data, clinical data, or genetic data.

Clause Q. The computer-implemented method of any of clause A to P, wherein the predetermined categorisation criterion is whether the image includes an animal.

Clause R. The computer-implemented method of any of clause A to Q, wherein the at least one input characteristic associated with the plurality of inputs comprises at least one of: individual response times for each of the plurality of inputs, a cumulative response time for the plurality of inputs, a mean response time, individual accuracy scores for each of the plurality of inputs, a cumulative accuracy score for the plurality of inputs or an overall accuracy score of the plurality of inputs.

Clause S. The computer-implemented method of clause R, further comprising determining a speed score of the plurality of inputs of the subject based on the input characteristic, wherein the input characteristic comprises the individual response times and the individual response times are within a predetermined range.

Clause T. The computer-implemented method of any of clauses R or S, further comprising determining a test index based on the overall accuracy score and the speed score.

Clause U. The computer-implemented method of claim T further comprising: calculating a plurality of predetermined statistical values of the test index, and determining an attention score based on the test index and the calculated statistical values of the test index.

Clause V. A system comprising: at least one processor and memory storing computer-executable instructions that, when executed by the one or more processors, cause the at least one processor to receive at least one set of response data comprising a plurality of inputs by a subject and an input characteristic associated with the plurality of inputs, wherein each of the inputs comprises a determination as to whether or not each image of a series of natural test images displayed satisfies at least one predetermined categorisation criterion; receive at least one set of characteristic data comprising at least one parameter indicative of a characteristic associated with the subject; process the at least one set of response data; process the at least one set of characteristic data; perform a first comparison by comparing the at least one set of processed response data to reference response data or to model reference data derived from reference response data; perform a second comparison by comparing the at least one set of processed characteristic data to corresponding reference characteristic data or to model characteristic data derived from corresponding reference characteristic data; and determine a status of at least one biomarker of a brain disorder in the brain of the subject based on the first comparison and the second comparison.

Clause W. A computer program product comprising instructions which, when the program is executed by a computer, cause the computer to carry out the method of any of clauses A to U.

Clause X. A computer-readable medium comprising instructions which, when executed by a computer, cause the computer to carry out the method of any of clauses A to U.

Clause Y. A method of treatment comprising: receiving at least one set of response data comprising a plurality of inputs by a subject and at least one input characteristic associated with the plurality of inputs, wherein each of the inputs comprises a determination as to whether or not each image of a series of natural test images displayed satisfies at least one predetermined categorisation criterion, receiving at least one set of characteristic data comprising at least one parameter indicative of a characteristic associated with the subject, processing the at least one set of response data, processing the at least one set of characteristic data, performing a first comparison by comparing the at least one set of processed response data to reference response data or to model reference data derived from reference response data, performing a second comparison by comparing the at least one set of processed characteristic data to corresponding reference characteristic data or to model characteristic data derived from corresponding reference characteristic data, determining a status of at least one biomarker of a brain disorder in the brain of the subject based on the first comparison and the second comparison, detecting an indication of the presence of at least one brain disorder based on the determined status of the at least one biomarker, and determining at least one therapeutic to be administered based on the detected indication of the at least one brain disorder, and administering said at least one therapeutic.

Clause Z. A method of obtaining an indication of an efficacy of the at least one therapeutic administered according to claim Z, the method comprising: performing the method according to any one of clauses A to U at a first time point and at a second time point for a given subject, wherein the therapeutic has been administered to the subject in an interval between the first time point and the second time point, performing a comparison of the status of the at least one biomarker of the brain disorder at the first time point to the status of the at least one biomarker of the brain disorder at the second time point, and determining, based on the comparison, the efficacy of the at least one therapeutic.

Clause AA. A method of treatment comprising: receiving at least one set of response data comprising a plurality of inputs by a subject and at least one input characteristic associated with the plurality of inputs, wherein each of the inputs comprises a determination as to whether or not each image of a series of natural test images displayed satisfies at least one predetermined categorisation criterion, receiving at least one set of characteristic data comprising at least one parameter indicative of a characteristic associated with the subject, processing the at least one set of response data, processing the at least one set of characteristic data, performing a first comparison by comparing the at least one set of processed response data to reference response data or to model reference data derived from reference response data, performing a second comparison by comparing the at least one set of processed characteristic data to corresponding reference characteristic data or to model characteristic data derived from corresponding reference characteristic data, determining a status of at least one biomarker of a brain disorder in the brain of the subject based on the first comparison and the second comparison, detecting an indication of the presence of at least one brain disorder based on the determined status of the at least one biomarker, and determining at least one non-pharmacological treatment to be implemented based on the detected indication of the at least one brain disorder.

Clause AB. A method of obtaining an indication of the efficacy of the at least one non-pharmacological treatment to be implemented according to clause AA, the method comprising: performing the method according to any one of clause A to U at a first time point and at a second time point for a given subject, wherein the non-pharmacological based treatment has been implemented in an interval between the first time point and the second time point, performing a comparison of the status of the at least one biomarker of the brain disorder at the first time point to the status of the at least one biomarker of the brain disorder at the second time point, and determining, based on the comparison, the efficacy of the at least one non-pharmacological based treatment.

Clause AC. A method for screening subjects, the method comprising: performing the method according to any one of clauses A to U for a plurality of subjects, performing, for each subject, a comparison of the status of the at least one biomarker of the brain disorder to the corresponding status of the at least one biomarker of the brain disorder in at least one other subject, and determining the risk of each subject of developing the at least one brain disorder based on each comparison.

Clause AD. A method for subject stratification, the method comprising: performing the method according to any one of clauses A to U for a plurality of subjects, performing, for each subject, a comparison of the status of the at least one biomarker of the brain disorder to the corresponding status of the at least one biomarker of the brain disorder in at least one other subject, and classifying, based on the comparison, each of the plurality of subjects into a plurality of sub-groups.

Clause AE. A method for determining a suitability of a treatment for a given candidate subject, the method comprising: performing the method according to any one of clauses A to U for the given candidate subject, and determining, based on the determined status of the at least one biomarker, the suitability of the treatment for the given candidate subject.

1. A computer-implemented method comprising:
receiving at least one set of pre-obtained response data comprising:
a plurality of inputs by a subject, wherein each of the inputs comprises a determination as to whether or not each image of a series of natural test images displayed satisfies at least one predetermined categorisation criterion; and
at least one input characteristic, wherein each input characteristic comprises data representative of at least one feature associated with one or more of the inputs;
receiving at least one set of pre-obtained characteristic data comprising at least one parameter indicative of a characteristic associated with the subject;
extracting the at least one input characteristic from the pre-obtained response data to generate at least one set of processed response data;
processing the at least one set of pre-obtained characteristic data to generate processed characteristic data;
performing a first comparison by comparing the at least one set of processed response data to corresponding reference response data or to model reference response data derived from corresponding reference response data, wherein the reference response data or the model reference response data is indicative of a status of at least one biomarker of a brain disorder;
performing a second comparison by comparing the at least one set of processed characteristic data to corresponding reference characteristic data or to corresponding model characteristic data derived from reference characteristic data, wherein the reference characteristic data or the model characteristic data is indicative of the status of the at least one biomarker of the brain disorder; and
determining the status of the at least one biomarker of the brain disorder in the brain of the subject based on the first comparison and the second comparison.

2. The computer-implemented method of claim 1 wherein the at least one set of pre-obtained response data comprises a plurality of sets of pre-obtained response data each obtained at a different respective time point.

3. The computer-implemented method of claim 2 wherein each different respective time point is separated by a predetermined time interval.

4. The computer-implemented method of any of claim 2 further comprising:
performing a third comparison by comparing each set of processed response data to each of the other respective sets of processed response data; and
determining a change in response data over time based on the third comparison,
wherein determining the status of the at least one biomarker of the brain disorder is further based on the determined change in response data.

5. The computer-implemented method of claim 1, wherein the status of the at least one biomarker of the brain disorder comprises at least one of biomarker positivity, concentration, volume or size of the at least one biomarker in the brain of the subject.

6. The computer-implemented method of claim 1 further comprising determining that the status of the at least one biomarker of the brain disorder exceeds a predetermined threshold.

7. The computer-implemented method of claim 1 further comprising determining that the status of the at least one biomarker of the brain disorder is lower than a predetermined threshold.

8. The computer-implemented method of claim 1 further comprising determining an indication of a risk of the subject developing at least one brain disorder based on the determined status of the at least one biomarker.

9. The computer-implemented method of claim 1 further comprising detecting an indication of the presence of at least one brain disorder based on the determined status of the at least one biomarker.

10. The computer-implemented method of claim 9 further comprising determining at least one therapeutic to be administered based on the detected indication of the at least one brain disorder.

11. The computer-implemented method of claim 1 further comprising determining at least one non-pharmacological treatment to be implemented based on the detected indication of the at least one brain disorder.

12. The computer-implemented method of claim 8 wherein the brain disorder is at least one of Alzheimer's Disease, cerebrovascular dementia, mild cognitive impairment, frontotemporal dementia, dementia with Lewy Bodies, multiple sclerosis, motor neurone disease, Parkinson's disease, attention deficit hyperactivity disorder, primary mental illness Huntington's disease, depression, post-traumatic stress disorder or brain injury.

13. The computer-implemented method of claim 1 wherein the at least one biomarker comprises at least one of neurofilament light chain, amyloid beta or Tau.

14. The computer-implemented method of claim 1, further comprising adding the at least one set of pre-obtained response data to the reference response data and the at least one set of pre-obtained characteristic data to the reference characteristic data once the biomarker status of the subject has been obtained.

15-17. (canceled)

18. The computer-implemented method of claim 1 wherein the at least one input characteristic associated with the plurality of inputs comprises at least one of: individual response times for each of the plurality of inputs, a cumulative response time for the plurality of inputs, a mean response time, individual accuracy scores for each of the plurality of inputs, a cumulative accuracy score for the plurality of inputs or an overall accuracy score of the plurality of inputs.

19. The computer-implemented method of claim 18 further comprising determining a speed score of the plurality of inputs of the subject based on the input characteristic, wherein the input characteristic comprises the individual response times and the individual response times are within a predetermined range.

20. The computer-implemented method of claim 18 further comprising determining a test index based on the overall accuracy score and the speed score.

21. The computer-implemented method of claim 20 further comprising:
calculating a plurality of predetermined statistical values of the test index; and
determining an attention score based on the test index and the calculated statistical values of the test index.

22. A system comprising:
at least one processor; and
memory storing computer-executable instructions that, when executed by the one or more processors, cause the at least one processor to:
receive at least one set of pre-obtained response data comprising a plurality of inputs by a subject and at least one input characteristic comprising data representative of at least one feature associated with one or more of the inputs, wherein each of the inputs comprises a determination as to whether or not each image of a series of natural test images displayed satisfies at least one predetermined categorisation criterion;
receive at least one set of pre-obtained characteristic data comprising at least one parameter indicative of a characteristic associated with the subject;
extract the at least one input characteristic from the pre-obtained response data to generate at least one set of processed response data;
process the at least one set of pre-obtained characteristic data;
perform a first comparison by comparing the at least one set of processed response data to corresponding reference response data or to corresponding model reference response data derived from reference response data, wherein the reference response data or the model reference response data is indicative of a status of at least one biomarker of a brain disorder;
perform a second comparison by comparing the at least one set of processed characteristic data to corresponding reference characteristic data or to corresponding model characteristic data derived from corresponding reference characteristic data, wherein the reference characteristic data or model characteristic data is indicative of the status of the at least one biomarker of the brain disorder; and
determine the status of the at least one biomarker of the brain disorder in the brain of the subject based on the first comparison and the second comparison.

23-24. (canceled)

25. A method of treatment comprising:
performing the method according to claim 10 and thereby determining that at least one therapeutic is to be administered; and
administering said at least one therapeutic.

26-31. (canceled)
